(19) United States
(12) Patent Application Publication
(10) Pub. No.: US 2023/0030702 A1
(43) Pub. Date: Feb. 2, 2023

(54) BLOCKING CHIMERIC ANTIGEN RECEPTORS FOR PREVENTION OF UNDESIRED ACTIVATION OF EFFECTOR AND REGULATORY IMMUNE CELLS

(71) Applicant: Migal Galilee Research Institute Ltd, Kiryat Shmona (IL)

(72) Inventors: Gideon GROSS, Moshav Almagor (IL); Noam LEVIN, Moshav Nahalal (IL); Hadas WEINSTEIN-MAROM, Kibutz Dafna (IL)

(21) Appl. No.: 17/640,319

(22) PCT Filed: Sep. 3, 2020
(86) PCT No.: PCT/IB2020/000710

§ 371 Date: Mar. 3, 2022

Related U.S. Application Data
(60) Provisional application No. 62/896,118, filed on Sep. 5, 2019.

(51) Int. Cl. C07K 14/705; C07K 14/725; C07K 16/28

(52) U.S. Cl. CPC C07K 14/70507; C07K 14/7051; C07K 14/70589; C07K 16/2833; C07K 14/70503; C07K 2319/03; C07K 2319/33; C07K 2319/50; C07K 2317/622; C07K 2317/569

(57) ABSTRACT

Provided herein are novel blocking chimeric antigen receptors (“bCARs”) and immune cells (e.g., effector and regulatory immune cells) that express such bCARs. Such blocking CARs prevent undesired activation of the immune cells, particularly undesired activation of the immune cells against normal tissue in therapeutic applications. Thus, such bCARs advantageously allow for selective immune cell activation only upon interaction with specific target cells (e.g., tumor cell).

### CROSS-REFERENCE TO RELATED APPLICATIONS

This application claims priority to U.S. Provisional Application No. 62/896,118, filed Sep. 5, 2019, which is herein incorporated by reference in its entirety.

### FIELD OF THE INVENTION

The invention relates to the use of immunotherapy by adoptive cell transfer for treatment of cancer or autoimmune disease, employing blocking antigen receptors to prevent undesired activation of effector and regulatory immune cells.

### BACKGROUND

Chimeric Antigen Receptor (CAR) T cell therapy, developed by the inventor almost three decades ago, emerges today as a most powerful approach for cancer treatment. Indeed, the FDA has recently approved the first CAR-based products for the treatment of certain types of non-Hodgkin lymphoma. Numerous research institutes and hundreds of companies worldwide (including Israel), with numbers constantly growing, all strive to develop safe and effective CAR T cell therapies for many types of cancers. Yet, a critical challenge faced by developers of CAR T cell therapies is the paucity of tumor-associated antigens that are widely shared by many patients and across many cancer types but are not expressed by any essential healthy tissue and can thus be targeted with no risk of on-target off-tumor toxicity. The most promising attempts to tackle this challenge aim at increasing the antigenic selectivity of the therapeutic CARs so as to avoid damage to non-tumor tissue, pursuing two complementary routes: 1) the use of inhibitory CARs (iCARs) to prevent CAR T cell activation in the presence of a “forbidden” antigen; 2) “combinatorial” recognition, devised to license CAR T cells to react only against antigenic signatures (comprising two antigens or more) which are entirely tumor-specific. Attempts have been made to develop “logic-gated” chimeric antigen receptor pairs which, when expressed by a cell, such as a T cell, are capable of detecting a particular pattern of expression of at least two target antigens. To date, none of these attempts, however, have proven feasible in the clinical setting. Thus, there remains a need for CAR based therapies that allow for antigenic selectivity and avoid damage to non-tumor tissue.

### BRIEF SUMMARY

Provided herein are novel blocking chimeric antigen receptors (“bCARs”) and immune cells (e.g., effector and regulatory immune cells) that express such bCARs. Such blocking CARs prevent undesired activation of the immune cells, particularly undesired activation of the immune cells against normal tissue in therapeutic applications. Thus, such bCARs advantageously allow for selective immune cell activation only upon interaction with specific target cells (e.g., tumor cell).

In a first aspect, provided herein is a polynucleotide encoding a blocking chimeric antigen receptor (bCAR) capable of preventing undesired activation of an immune cell. The bCAR includes an extracellular domain, a transmembrane domain and an intracellular domain. The extracellular domain that is more than about 20 nm in length and includes: a single-chain binding domain; a rigid elongation domain, and a protease cleavage site capable of being cleaved by a protease. The intracellular domain includes a full or partial intracellular domain of a membrane phosphatase capable of dephosphorylating phosphorylated immunoreceptor tyrosine-based activation motifs (ITAMs). The protease cleavage site is linked to the intracellular domain by the transmembrane domain. The binding domain specifically binds to a cell surface epitope present on normal mammalian tissue, but absent on related abnormal mammalian tissue or mammalian tissue being targeted by an autoimmune response.

In some embodiments, the protease is a disintegrin and metalloproteinase (ADAM) or a beta-secretase 1 (BACE1). In exemplary embodiments, the protease cleavage site comprises Lin 12/Notch repeats and/or an ADAM protease cleavage site. In certain embodiments, the protease is a disintegrin and metalloproteinase (ADAM) or a beta-secretase 1 (BACE1).

In another aspect, provided herein is a polynucleotide that encodes an elongated blocking chimeric antigen receptor (bCAR) capable of preventing undesired activation of an immune cell. The bCAR includes an extracellular domain that is more than about 20 nm in length; a transmembrane domain; and an intracellular domain. The extracellular domain includes a single-chain binding domain, and a rigid elongation domain. The an intracellular domain includes a module capable of association or colocalization with T cell receptor (TCR)-CD3 complex. The elongation domain is linked to the intracellular domain by the transmembrane domain. The binding domain specifically binds to a cell surface epitope present on normal mammalian tissue but not on related abnormal mammalian tissue or mammalian tissue being targeted by an autoimmune response. In some embodiments, the module capable of association or colocalization with T cell receptor (TCR)-CD3 complex is the intracellular domain of native CD2.

In some embodiments provided herein, the binding domain comprises (i) a single chain variable fragment (scFv); a functional fragment of an antibody; a single-domain antibody, such as a Nanobody; and a recombinant antibody; (ii) an antibody mimetic, such as an affibody molecule; an affilin; an affimer; an affitin; an alphabody; an anticalin; an avimer; a DARPin; a fynomer; a Kunitz domain peptide; and a monobody; (iii) an aptamer; or (iv) a binding domain of a native receptor, such as a cytokine receptor or a ligand such as a cytokine.

In some embodiments, the membrane phosphatase capable of dephosphorylating phosphorylated ITAMs is CD45 or CD148.

In certain embodiments, the rigid elongation domain comprises at least one rigid protein module. In exemplary embodiments, the at least one rigid protein module comprises a fibronectin type III repeat or an Ig domain comprising an Ig fold motif. In some embodiments, the at least one rigid protein module comprises the extracellular domain of CD45 or CD148 or a fragment thereof. In certain embodiments, the rigid elongation domain is linked to the extracellular or transmembrane domain of CD2, CD4, or CD8, or a fragment thereof. In some embodiments, the elongation domain is linked to the extracellular and/or transmembrane domain of CD2. In certain embodiments, the abnormal tissue is a pre-malignant tissue or a malignant tumor.

In some embodiments, the cell surface epitope is a single allelic variant of a polymorphic cell surface epitope not expressed by the abnormal mammalian tissue but present on all cells of related mammalian normal tissue. In certain embodiments, the cell surface epitope is a cell surface epitope of an essential tissue-associated gene product or housekeeping gene products. In some embodiments, the cell surface epitope is an ion channel or a receptor tyrosine kinase.

In exemplary embodiments, the mammalian tissue being targeted by an autoimmune response is selected from the group consisting of pancreatic islets of the pancreas; digestive system; small intestine; large intestine; colon; thyroid; nervous system; skin, thyroid, and articular joints. In some embodiments, the mammalian tissue is human tissue.

In exemplary embodiments, the undesired activation of an immune cell is activation caused by specific binding of the immune cell to normal tissue via one or more cell-surface epitopes present on normal and related abnormal mammalian tissue or mammalian tissue being targeted by an autoimmune response. In some embodiments, the immune cell is an effector immune cell, such as an effector T cell; or a regulatory immune cell, such as a regulatory T cell.

In exemplary embodiments, the binding domain is an scFv; the elongation domain comprises an extracellular domain of a CD45 or a CD148 or fragment thereof the elongation domain comprises a plurality of fibronectin repeats, and the elongation domain is optionally linked to the an extracellular domain or transmembrane domain or fragment thereof of CD2, CD4 or CD8.

In exemplary embodiments, the binding domain is an scFv, and the protease is a disintegrin and metalloproteinase (ADAM) or a beta-secretase 1 (BACE1).

In some embodiments, the binding domain is a scFv and the intracellular domain is an intracellular domain of CD45, CD148 or CD2 or fragment thereof.

In some embodiments, the binding domain is an scFv, the elongation domain comprises an extracellular domain of a CD45 or a CD148 or fragment thereof, the elongation domain comprises a plurality of fibronectin repeats, and the protease is a disintegrin and metalloproteinase (ADAM) or a beta-secretase 1 (BACE1).

In some embodiments, the binding domain is an scFv; the elongation domain comprises an extracellular domain of a CD45 or a CD148 or fragment thereof, the elongation domain comprises a plurality of fibronectin repeats, the elongation domain is optionally linked to a CD2 extracellular domain or transmembrane domain or fragment thereof, and intracellular domain is a CD45, CD148 or CD2 intracellular domain or fragment.

In certain embodiments, the binding domain is an scFv, the elongation domain comprises an extracellular domain of a CD45 or a CD148 or fragment thereof, the elongation domain comprises a plurality of fibronectin repeats, the protease is a disintegrin and metalloproteinase (ADAM) or a beta-secretase 1 (BACE1), and the intracellular domain is a CD45 or CD148 intracellular domain or fragment thereof.

In some embodiments, the binding domain is an scFv, the elongation domain comprises a plurality of fibronectin repeats, the elongation domain is linked to a CD2 extracellular domain or transmembrane domain or fragment thereof, and the intracellular domain is a CD2 intracellular domain.

In another aspect, provided herein is a polynucleotide encoding a short blocking chimeric antigen receptor (sbCAR) capable of preventing undesired activation of an immune cell. The sbCAR includes an extracellular domain; an elongation domain and a transmembrane domain and an intracellular domain. The extracellular domain is less than about 20 nm in length and includes a single-chain binding domain, and an elongation domain. The intracellular domain comprising a full or partial intracellular domain of a membrane phosphatase capable of dephosphorylating phosphorylated immunoreceptor tyrosine-based activation motifs (ITAMs). The undesired activation of the immune cell is activation caused by specific binding of the immune cell to normal tissue via one or more cell-surface epitopes present on normal and abnormal mammalian tissue or mammalian tissue being targeted by an autoimmune response. The binding domain specifically binds to another cell surface epitope present on abnormal mammalian tissue or mammalian tissue being targeted by an autoimmune response but not on normal mammalian tissue. The undesired activation of the immune cell is activation caused by specific binding of the immune cell to normal tissue via one or more cell-surface epitopes present on normal and abnormal mammalian tissue or mammalian tissue being targeted by an autoimmune response. The total length of the extracellular domain and the cell surface epitope is more than about 20 nm.

In some embodiments, the elongation domain comprises a fibronectin type III repeat. In certain embodiments, the elongation domain comprises a partial extracellular domain of CD45 or CD148 or an Ig-domain. In some embodiments, the ITAMs is CD45 or CD148.

In exemplary embodiments, the undesired activation of an immune cell is activation caused by specific binding of the immune cell to normal tissue via a first cell-surface epitopes present on normal and related abnormal mammalian tissue or related mammalian tissue being targeted by an autoimmune response, wherein the undesired activation is prevented by specific binding of the binding domain of the sbCAR to a second cell surface epitope which in combination with the first cell surface epitope is present on abnormal tissue or mammalian tissue being targeted by an autoimmune response but not on related normal tissue.

In another aspect, provided herein are expression vectors that include any of the polynucleotides provided herein and at least one control element operably linked to the polynucleotide.

In another aspect, provided herein is an effector immune cell, such as an effector T cell, that specifically binds to and is potentially activated by a first cell surface epitope shared by cells of related abnormal and normal mammalian tissue, wherein the effector immune cell comprises: a) any of the subject polynucleotides provided herein that encodes a bCAR herein, wherein the bCAR specifically binds to a second cell surface epitope present on normal mammalian tissue but not on related abnormal mammalian tissue; or b) an expression vector comprising the polynucleotides.

In another aspect, provided herein is an effector immune cell, such as an effector T cell, that specifically binds to and is potentially activated by a first cell surface epitope shared by cells of related abnormal and normal mammalian tissue, wherein the effector immune cell comprises a) any of the subject polynucleotides that encodes a sbCAR provided herein, wherein the sbCAR specifically binds to a second cell surface epitope that in combination with the first cell surface epitope is present on abnormal tissue but not on related normal tissue, or b) an expression vector comprising the polynucleotide. In certain embodiments, the effector immune cell further includes a polynucleotide encoding an activating chimeric antigen receptor (aCAR) polypeptide providing the specific binding to, and potential activation by, the first cell surface epitope.

In some embodiments, a regulatory immune cell, such as a regulatory T cell, that specifically binds to and is potentially activated by a cell surface epitope on a mammalian tissue being targeted by an autoimmune response, wherein the regulatory immune cell comprises: a) any of the subject polynucleotides provided herein that encodes a bCAR provided herein, wherein the bCAR specifically binds to a second cell surface epitope present on normal mammalian tissue but not on related abnormal mammalian tissue; or b) an expression vector comprising the polynucleotide.

In another aspect, provided herein is a method for treating cancer comprising administering to a cancer patient immune effector cells provided herein. In some embodiments, the immune effector cells are T cells, natural killer cells or cytokine-induced killer cells. In exemplary embodiments, the immune effector cells are universal allogeneic effector cells.

In another aspect, provided herein is a method of treating an autoimmune disease comprising administering to an autoimmune disease patient any of the regulatory immune cells provided herein. In some embodiments, the regulatory immune cells are universal (allogeneic) effector cells, such as universal regulatory T cells.

Also provided herein are bCARs encoded by the subject polynucleotides provided herein.

### BRIEF DESCRIPTION OF THE DRAWINGS

FIG. 1 depicts a schematic of the kinetic-segregation (KS) model for T cell activation. In a resting T cell (a), random protein interactions in the membrane lead to phosphorylation and dephosphorylation of molecules with tyrosine-phosphorylation motifs by Src kinases and tyrosine phosphatases. Triggering occurs as the local balance of those constitutive processes is altered by the formation of close-contact zones (b-d). This process is “nucleated” by small proteins such as CD2 and results in the local size-dependent exclusion of large proteins such as CD45 from the close-contact zone. The “fates” of the three TCRs here (1, 2, 3) are presented according to the KS model. In the absence of peptide-MHC contact, TCR 1 diffuses from the contact zone. If TCR 1 had been phosphorylated in the close-contact zone or before its formation, rapid dephosphorylation outside the close-contact zone would prevent signaling. TCR 2, phosphorylated before close contact zone formation, binds cognate MHC-peptide and is thereby “held” in the close-contact zone long enough for “downstream” events to occur. TCR 3, phosphorylated after close-contact zone formation, is phosphorylated by free Lck only, accounting for coreceptor-independent triggering. Relatively large amounts of phosphorylation of TCR 2 and TCR 3 lead to “downstream” signaling after Zap70 recruitment.

FIG. 2 are schematics depicting the dimensions of molecules involved in T cell antigen recognition. (FIG. 2A) A schematic view of the dimensions of cell-surface molecules involved in T cell antigen recognition. Sizes are estimated from structural studies of these or related molecules with the exception of CD148. (FIG. 2B) The kinetic segregation model of TCR triggering and immunological synapse formation.

FIG. 3 is a schematic, showing that size-dependent sorting mechanism operates at local zones of contact between T cells and antigen-presenting cells.

FIG. 4 provides a schematic, showing the contrasting effects of wild-type and elongated CD48 on T cell antigen recognition. (FIG. 4A) CD2 molecules on T cells and CD48 molecules on I-EK1 CHO APCs interact to form contacts in which the intermembrane separation distance is determined by the dimensions of the CD2/CD48 complex. Wild-type CD48 (left) enhances T cell antigen recognition because the separation distance (≈15 nm) is optimal for TCR engagement of peptide-MHC. Elongated CD48 (CD48-CD22, (right) inhibits T cell antigen recognition by forming contacts in which the intermembrane distance (>20 nm) is too great for TCR to engage pMHC. (FIG. 4B). Elongated forms of CD48 inhibit T cell antigen recognition. Antigen recognition by 2B4.CD2 cells using as APCs CHO cells expressing no CD48 (CD48 neg CHO), wild-type CD48 (CD48 CHO), CD48-CD2, or CD48-CD22.

FIG. 5 depicts the molecular design and mode of action of bCARs provided herein. (FIG. 5A) A schematic representation of the bCARs underlying the three strategies proposed herein. The CD2-CARs are shown in two configurations: (I) possessing additional Ig-like domains; (II) with the ectodomain of CD45. (FIG. 5B). Engagement of the aCAR by target cells expressing the activating antigen but not the protective one will result in exclusion of both native CD45 and the CD45-CAR, as postulated by the KS model, and lead to T cell activation. (FIG. 5C). Engagement with non-target cells to be protected which co-express the activating and the protective antigens allows bCAR in the contact zone. In this scenario, the bCAR functions as a “spacer clamp”, thereby preventing binding of the compact aCAR to its target antigen while its phosphatase activity is retained in close proximity to any phosphorylated aCAR that may be present locally. (FIG. 5D). Upon binding to the protective antigen, CD2-CAR is expected to extent the same steric effect as does CD45-CAR, while the phosphatase activity of non-excluded native CD45 will further prevent activation. (FIG. 5E) The anticipated mode of action of the CD45N/CAR. Following antigen binding, protein cleavage will liberate the ectodomain while retaining the short CD45 transmembrane and phosphatase domains at the contact zone. Under such circumstances, ITAM phosphorylation will be counteracted by the phosphatase activity and local activation will be blocked. (FIG. 5F). sCD45-CAR. The core component comprises a truncated ectodomain, the transmembrane region and the intracellular phosphatase portion of CD45, all attached to the binding moiety (here scFv). The truncated ectodomain can alternatively be derived from other rigid scaffolds of human cell surface proteins and its optimal size will be determined experimentally, based on current data on the dimensions of the respective components.

FIG. 6 provides a summary of several exemplary bCARs described herein. (FIG. 6A) Upper panel, left: schemes of the eleven constructs which are described herein. Upper panel, right: the different components incorporated into these CD2-bCARs. Lowe panel: mRNA was synthesized in-vitro form the eleven templates. Shown is agarose gel electrophoresis of small samples of all these mRNAs. (FIG. 6B) Flow cytometry analysis for cell surface expression of the products of these mRNAs following electroporation of K562 cells, using the anti-Myc tag mAb. N.C., negative control (pulsed cells only with no mRNA); P.C., positive control (cells transfected with mRNA encoding the anti-A2 CAR). Sample 1494 was defective and is not included in this figure.

FIG. 7 provides a summary of an experiment, showing that several exemplary bCARs are capable of inhibiting antigen-dependent aCAR-induced T cell activity in an antigen-dependent manner. Percent inhibition of T cell activation was calculated relative to the activation of B3Z cells transfected with irrelevant mRNA in the presence of peptide-loaded RMA-A2 vs. RMA cells.

FIG. 8 provides a summary of another experiment, showing that several exemplary bCARs are capable of inhibiting antigen-dependent aCAR-induced T cell activity in an antigen-dependent manner. Percent inhibition of T cell activation was calculated relative to the activation of B3Z cells transfected with irrelevant mRNA in the presence of peptide-loaded RMA-A2 vs. RMA cells.

FIG. 9 provides a summary of another experiment, showing that several exemplary bCARs are capable of inhibiting antigen-dependent aCAR-induced T cell activity in an antigen-dependent manner using human Jurkat T cells expressing anti-melanotransferrin aCAR and anti-HLA-A2 bCAR. Results are presented as relative luminescence units (RLU). Controls: Nonpul, non-pulsed Jurkat cells; Irr, 20 mg of irrelevant mRNA. % inhibition was calculated as (% inhibition with 579-A2)−(% inhibition with 579) where luciferase activity of cells transfected with aCAR+Irr mRNA against 579-A2 and against 579 cells, respectively, is considered 100% activity and activity of transfected cells with no melanoma cells is background value.

FIGS. 10A and B provide an assessment of the inhibitory capacity of exemplary bCARs described herein. NFAT-Luc reporter Jurkat cells were co-electroporated with mRNA encoding the anti-MTf aCAR and the indicated anti-A2 bCARs or irrelevant mRNA (Irr.) at 1:4 mRNA ratio. Cells were then co-incubated with 579 or 579-A2 melanoma cells and cell lysates were then assayed for luciferase activity using a luminometer. (FIGS. 10A and B show the results of two independent experiments. RLU, relative luminescence units. Percent inhibition was calculated relative to the RLU value recorded for Irr. mRNA, where Irr. mRNA only serves as background. 1558 is a conventional anti-A2 activating CAR harboring the same scFv and served control for T cell activation.

FIGS. 11 and B depict a summary of additional bCARs. FIG. 11A. Scheme of the new series of bCARs. The anti-HLA-A2 scFv has been replaced with that of the anti-melanotransferrin (MTf) CAR. A hemagglutinin (Ha) tag is placed c-terminally to the scFv. FIG. 11B. Flow cytometry analysis for iCAR expression following electroporation of K562 cells using an anti-Ha tag mAb. Black histograms, irrelevant mRNA. Old and new numerical designations are shown.

FIG. 12 depicts a study assessing the inhibitory capacity of bCAR 1766. Reporter NFAT-Luc Jurkat cells were electroporated with the indicated mixtures of aCAR, iCAR or irrelevant (Irr.) mRNA. Cells were then co-incubated with 579-A2 melanoma cells and cell lysates were assayed for luciferase activity using a luminometer. RLU, relative luminescence units. Percent inhibition was calculated relative to the RLU value recorded for Irr. mRNA.

FIGS. 13A-D depict a summary of characterization studies of additional bCARs that inhibit T cell activation specific manner. (FIG. 13A) Schemes of the bCARs tested and their designation. bCARs are directed against MTf and carry the Ha Tag. (FIGS. 13B and C) Inhibition assay. Reporter NFAT-Luc Jurkat cells were electroporated with the indicated mixtures of aCAR, bCAR or irrelevant (Irr.) mRNA. Cells were then co-incubated with 579-A2 melanoma (FIG. 13B) or RMA-A2 cells (FIG. 13C) and cell lysates were assayed for luciferase activity using a luminometer. RLU, relative luminescence units. (FIG. 13D) Percent inhibition was calculated relative to the RLU value recorded for 1558+Irr. mRNA in each coculture. CAR 1656 is another anti-HLA-A2 aCAR, which served as a control.

FIGS. 14A-D depict a summary of studies of iCAR with CD2 mutant endodomains to uncouple signaling activity from cytoskeletal binding. A schematic representation of these mutants and their designations are shown in FIG. 14A. All these constructs harbor an anti-MTf scFv and the Ha tag and are based on either the original 1495 or 1498 iCAR constructs and mutations and designations are specified (FIG. 14A). High surface expression of all these constructs following mRNA transfection of K562 cells is shown in FIG. 3B. The ability of the different mutants to inhibit the activation of NFAT-Luciferase reporter Jurkat cells co-transfected with an a CAR specific to HLA-A2 (1558) upon coculture with RMA-A2 cells (MTf(−)) or 579-A2 melanoma cells (MTf(+) is shown in FIG. 14C. While no clear inhibition was evident for the iCARs in the absence of the inhibitory antigen (the MTf(−) RMA-A2 cells) inhibition ranging between ˜40% and 55% was evident for all of the iCARs tested. No reproducible effect could be ascribed to the two mutations or their combinations as examined here. Of note, an unexpected response of Jurkat cells expressing the 1656 aCAR against the MTf(−) RMA-A2 cells could be observed.

FIG. 15 provides a schematic of an exemplary mechanism of action of the SynNotch-based bCARs. The activating antigen appears as a turquoise ellipse and the inhibitory antigen as a yellow triangle.

FIG. 16 Scheme of the SynNotch-based bCARs already constructed in this study. See legends to FIG. 6 and FIG. 15 for additional details.

FIG. 17 shows flow cytometry analysis for construct expression. HEK293T cells were transiently transfected with plasmid DNA of a selected SynNotch bCAR or empty pBJ1-Neo vector using FuGENE HD transfection reagent (Promega). Fluorescence was analyzed at the indicated time points post-transfection.

FIG. 18 shows flow cytometry analysis of HEK293T and COS7 cells for the expression of HLA-A2. Staining was performed with non-conjugated BB7.2 mAb and secondary goat-anti mouse Abs. 579 and 579-A2 melanoma cells were used a negative and positive controls, respectively.

FIGS. 19A-H depict exemplary extracellular components that can be used in any of the subject bCARs provided herein.

FIG. 20 depict exemplary transmembrane domains that can be used in any of the subject bCARs provided herein.

FIGS. 21A and B depict exemplary intracellular domains that can be used in any of the subject bCARs provided herein.

FIGS. 22A and B depict additional exemplary sequences that can be used in any of the subject bCARs provided herein, including the Notch core regulatory region of the SynNotch receptor and an exemplary HLA-A2 scFv.

### DETAILED DESCRIPTION

### I. Overview

Provided herein are novel blocking chimeric antigen receptors (“bCARs”) and immune cells (e.g., effector and regulatory immune cells) that express such bCARs. Such blocking CARs prevent undesired activation of the immune cells, particularly undesired activation of the immune cells against normal tissue in therapeutic applications. Thus, such bCARs advantageously allow for selective immune cell activation only upon interaction with specific target cells (e.g., tumor cell).

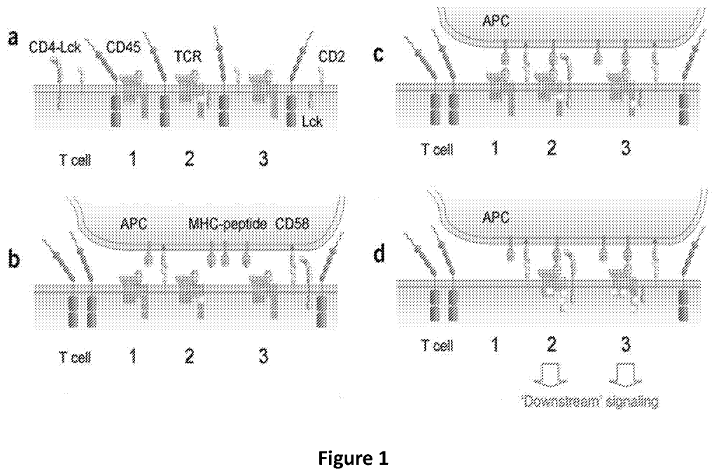

The bCARs provided herein capitalize on the kinetic-segregation (KS) model for T cell activation by an antigen presenting cell or a target cell (APC/T) (FIG. 1). Ligation of the TCR by peptide/MHC (pMHC) complexes on APC/T triggers activation signaling. One of the earliest events in this process is the phosphorylation of tyrosine residues in the immune-receptor-tyrosine-based-activation-motifs (ITAMs) of the TCR CD3 γ, δ, ε and ζ subunits, mainly by the Src family nonreceptor tyrosine kinase Lck, which is noncovalently associated with the CD4 and CD8 coreceptors. This step, in turn, activates the ZAP70 protein tyrosine kinase, leading to phosphorylation of downstream adapter proteins and enzymes and, eventually, to the transmission of the integrated signals into the T cell nucleus. CD45 is an abundant cell surface protein tyrosine phosphatase with exceptionally high catalytic activity that plays a critical role in the regulation of T cell activation. Prior to encountering antigen, CD45 dephosphorylates a C-terminal negative regulatory tyrosine on Lck, potentiating this kinase to readily phosphorylate CD3 ITAMs upon TCR ligation. The demonstrations that global phosphatase inhibitors and kinase activators can induce spontaneous T cell activation in the absence of antigen have prompted the notion that CD45 serves as a safeguard, preventing non-specific T cell activation by maintaining a sub-threshold level of phosphorylated ITAMs. This scenario immediately raised the question as to how CD45 activity is prevented considering the high rate of ITAM phosphorylation which follows TCR ligation, as CD45 cannot discriminate between “legitimate” and “prohibited” phosphotyrosines. The KS model provides a mechanistic explanation for the regulation of TCR signaling by CD45 and has received ample experimental support since first introduced.

Following are distinct features of the KS model which directly pertain to the new concept:

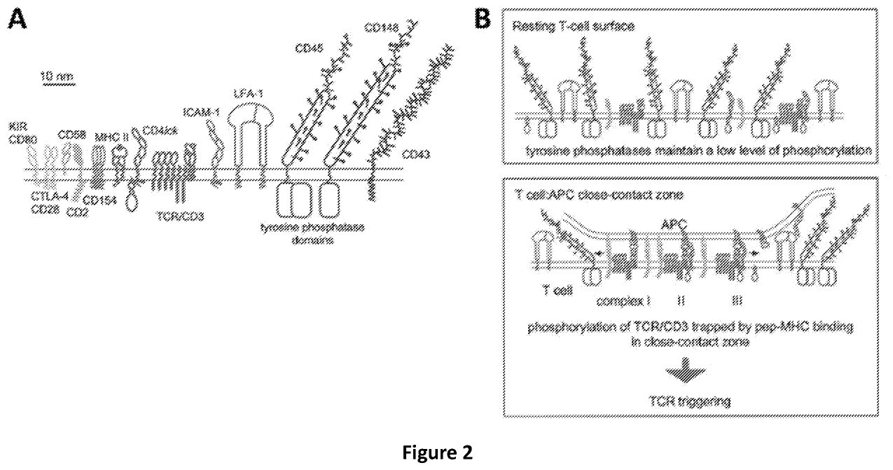

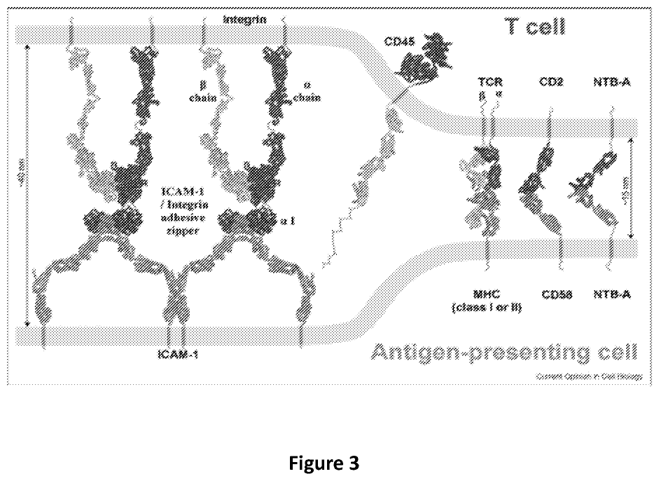

The close contact zone that initially forms between the two cells is primarily occupied with compact “binding” cell surface molecules, including the TCR, CD4/CD8, CD28, CD2 and SLAMF6 on the T cell and pMHC-II, B7, CD58 (LFA3, CD48 in the mouse) and SLAMF6 on the APC/T, creating an interface of ≈15 nm. To allow these interactions, bulky T cell surface molecules, including CD45, CD148, CD43 and LFA1, some spanning 40 nm and more, are excluded from the contact zone (FIGS. 2, 3).

In the periphery of the contact zone, T cell-APC/T interactions are stabilized by the formation of zipper-like complexes between T cell integrins (e.g., LFA1) and cell adhesion molecules (such as ICAM-1) on the interacting cells (FIG. 3). Sorting of large integrins to these designated areas is governed by the actin cytoskeleton so that separation between narrow antigen-specific interfaces and wide non-specific ones guarantees that T cell signaling is not sterically hindered.

The exclusion of CD45 and CD148 from the contact zone is critical for TCR signaling. An important structural component of the CD45 ectodomain which confers the rigidity necessary for exclusion comprises three fibronectin type III repeats. The expression of truncated forms of these phosphatases prevented their exclusion and resulted in strong inhibition of T cell activation.

The exclusion of elongated chimeric Lck from the contact zone prevented T cell activation, corroborating the importance of molecular dimensions and size-based sorting for T cell signaling.

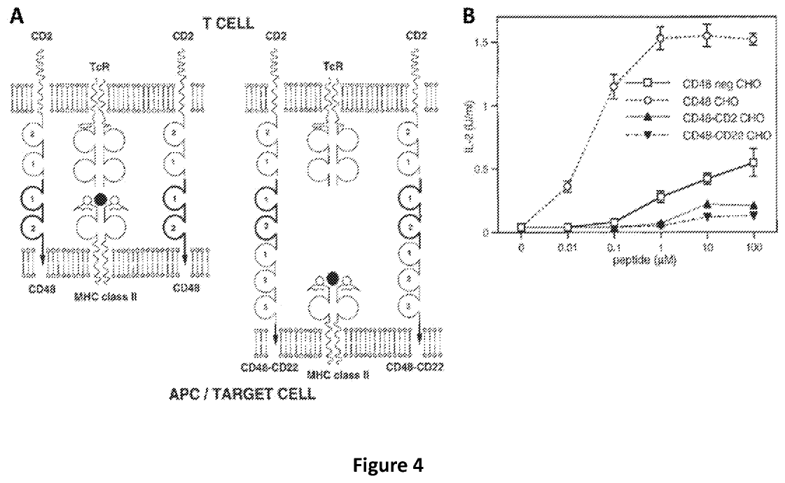

The CD2 adhesion molecule physically associates with the TCR-CD3 complex at the T cell surface and plays a major role in cytoskeletal polarization in the contact zone with APC/T. Artificially elongated derivatives of the CD2-CD48 axis (achieved via genetic engineering of the CD48 ligand) prevented TCR-mediated signaling in a T cell hybridoma and severely reduced proliferation of primary T cells in response to TCR stimulation. This inhibitory effect could be attributed to the increased intermembrane spacing, which was too wide to accommodate TCR-pMHC interactions (FIG. 4). It is proposed that reorganization of the immunological synapse enforced by the extended CD48 ectodomain sequestered the TCR in a location where it could no longer interact with pMHC, dramatically reducing T cell sensitivity.

Similarly to CD2-CD48, elongation of the TCR-pMHC axis through incremental extensions of the pMHC ectodomain almost completely blocked TCR triggering without affecting TCR-pMHC ligation, an effect that was ascribed to increased retention of CD45 at the contact zone.

The height of a surface protein above the cell membrane, rigidity of its ectodomain, the area of its lateral footprint on the membrane, the presence of a ligand on the opposing membrane, the binding affinity for such a ligand and the membrane density of the protein were all deduced to be critical factors which govern presence in, or exclusion from, the contact zone that is formed by binding proteins. Importantly, crowding of binding proteins contributed to exclusion of non-binding ones which, size-wise, could still occupy the interface. Furthermore, the “exclusion threshold” was sharp and additional extensions of a non-binding protein above this threshold had a negligible effect on exclusion.

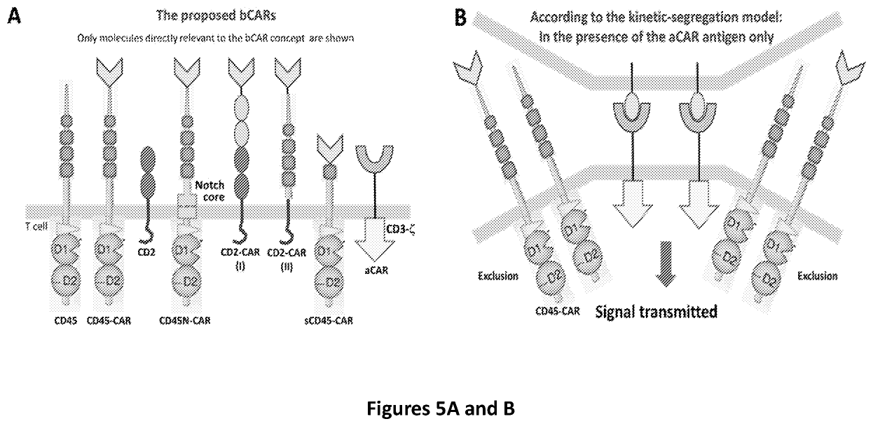

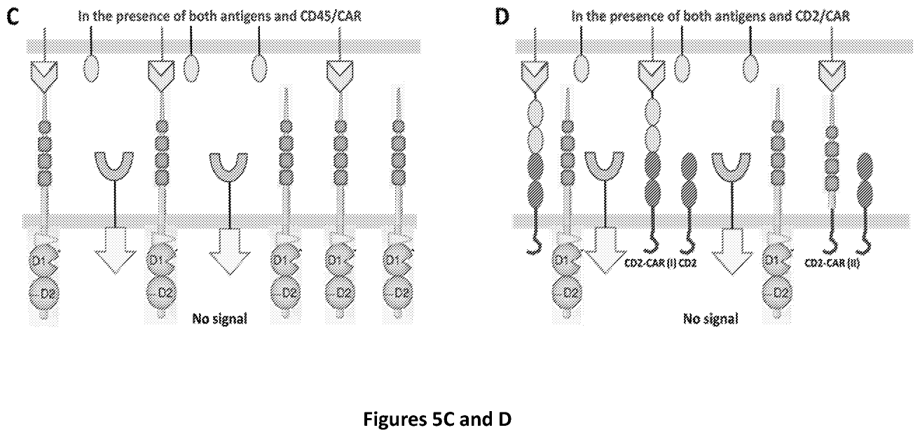

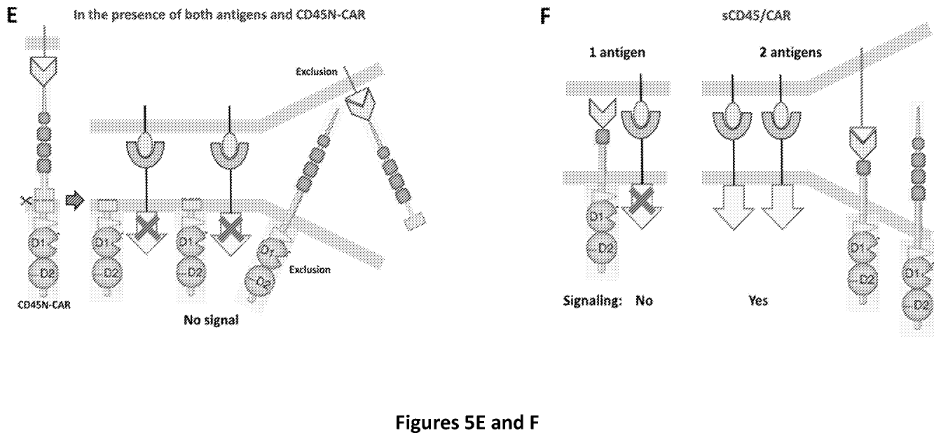

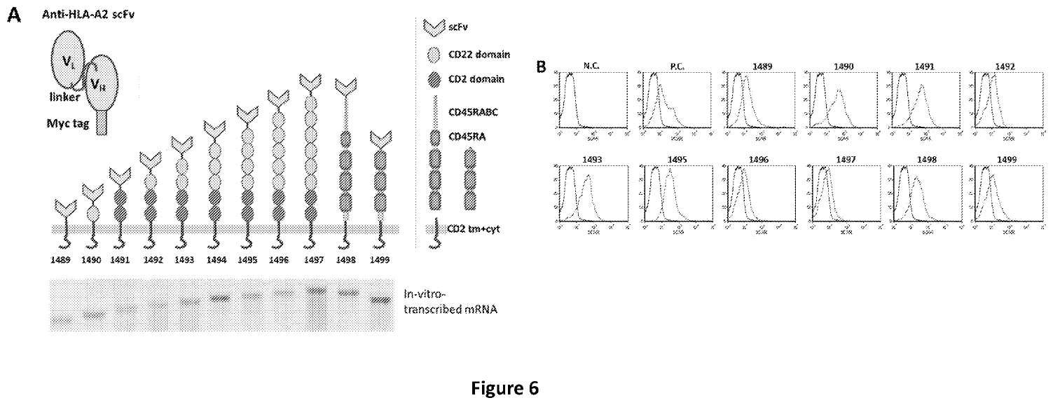

The present invention, based on the KS model, provides for improved blocking CARs (see, e.g., FIG. 5A and FIG. 6A), including elongated blocking CARs, CD45N-CARs, and “short” sCD45-CARs. Aspects of each of these CARs are described in further detail below.

### II. Definitions

The terms “treat”, “treating”, “treatment”, etc., as applied to an isolated cell, include subjecting the cell to any kind of process or condition or performing any kind of manipulation or procedure on the cell. As applied to a subject, the terms refer to administering a cell or population of cells in which a target polynucleotide sequence (e.g., B2M) has been altered ex vivo according to the methods described herein to an individual. The individual is usually ill or injured, or at increased risk of becoming ill relative to an average member of the population and in need of such attention, care, or management.

As used herein, the term “treating” and “treatment” refers to administering to a subject an effective amount of cells with target polynucleotide sequences altered ex vivo according to the methods described herein so that the subject has a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a disorder associated with expression of a polynucleotide sequence, as well as those likely to develop such a disorder due to genetic susceptibility or other factors.

By “treatment” or “prevention” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

In some embodiments, the alteration results in reduced expression of the target polynucleotide sequence. The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

Before the invention is further described, it is to be understood that this invention is not limited to particular embodiments described, as such may, of course, vary. It is also to be understood that the terminology used herein is for describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

### III. Detailed Description of the Embodiments

A. Elongated bCARs

In one aspect, provided herein are elongated bCARs. Such elongated bCARs expressed on an immune cell (e.g., effector and regulatory cells) are capable of binding to a first antigen on a target cell and creating a widened contact zone that prevents an activation CAR (aCAR) or TCR coexpressed on the immune cell from binding a second antigen expressed on a target cell (see, e.g., FIGS. 5C and D). Thus, in some embodiments, such immune cells that coexpress both aCARs and bCARs are capable of activation upon binding to target cells that express the antigen recognized by the aCAR alone (see FIG. 5B), but are hindered from activation in the presence of target cells that express the bCAR target antigen (see FIGS. 5C and D). Such elongated bCARs advantageously allow for the selective activation of an immune cell against a desired target cell (e.g., a tumor cell or a target of an autoimmune reaction) and reduces undesired immune cell activation (e.g., activation against non-tumor cells).

In some embodiments, the bCAR is an elongated bCAR that includes an extracellular domain of more than about 20 nm in length. The extracellular domain includes a single chain binding domain, and a rigid elongation domain. In some embodiments, the single chain binding domain binds to a cell surface molecule expressed on a target cell to be protected. Such bCARs also include an intracellular domain linked to the extracellular domain via a transmembrane domain. The intracellular domain includes a module capable of association or colocalization with T cell receptor (TCR)-CD3 complex.

In some embodiments, the elongated bCAR is co-expressed in an immune cell (e.g., a T cell) that includes an activating CAR (aCAR, a “conventional” CAR) or a TCR. In certain embodiments, the length of the extracellular domain of the bCAR is longer than the distance of the aCAR/antigen and/or TCR/antigen complex. As the length of the extracellular domain of the bCAR is longer than the distance of the aCAR/antigen and/or TCR/antigen complex, such bCARs, when bound to its antigen on a target cell to be protected, forms a “spacer clamp” that prevents physical contact between a natural TCR or an activating CAR (aCAR, a “conventional” CAR) with their respective antigens (as shown in FIGS. 4, 5C). Furthermore, the wide interface enforced by the elongated bCAR also prevents exclusion of native CD45 in the contact zone, adding substantial protection from undesired signaling.

In some embodiments, the extracellular domain is more than 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 nm in length. In exemplary embodiments, the extracellular domain is more than about 20 nm in length.

Any suitable single chain binding domain can be included in the extracellular domain that allows for binding to an epitope of a target cell. Suitable binding domains that can be incorporated in the bCARs (e.g., the elongated bCARs) provided herein include, but are not limited to: a single chain variable fragment (scFv), a functional fragment of an antibody, a single-domain antibody (e.g., a nanobody), a recombinant antibody or a fragment thereof. In some embodiments, the binding domain is an antibody mimetic, such as an affibody molecule; an affilin; an affimer; an affitin; an alphabody; an anticalin; an avimer; a DARPin; a fynomer; a Kunitz domain peptide; and a monobody. In certain embodiments, the binding domain of the bCAR is an aptamer. In some embodiments, the binding domain is a binding domain of a native receptor, such as a cytokine receptor or a ligand such as a cytokine.

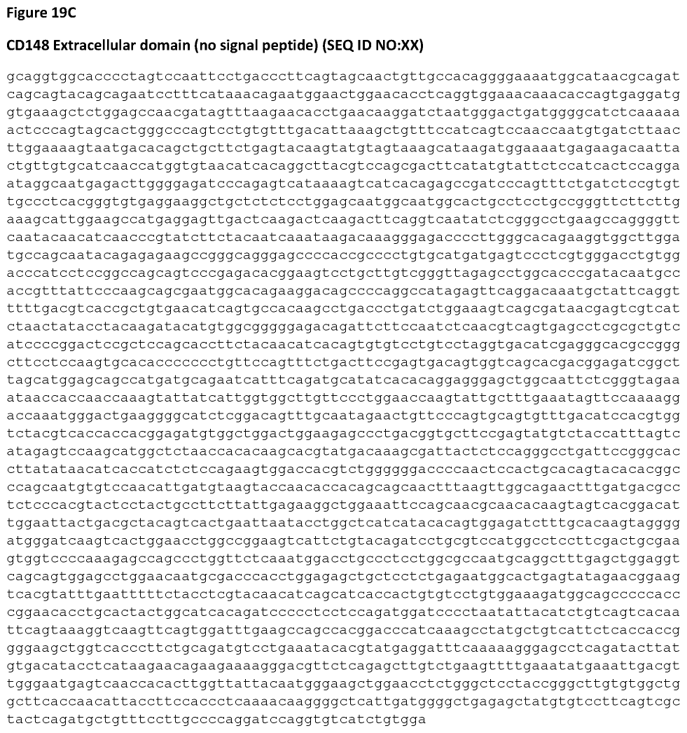

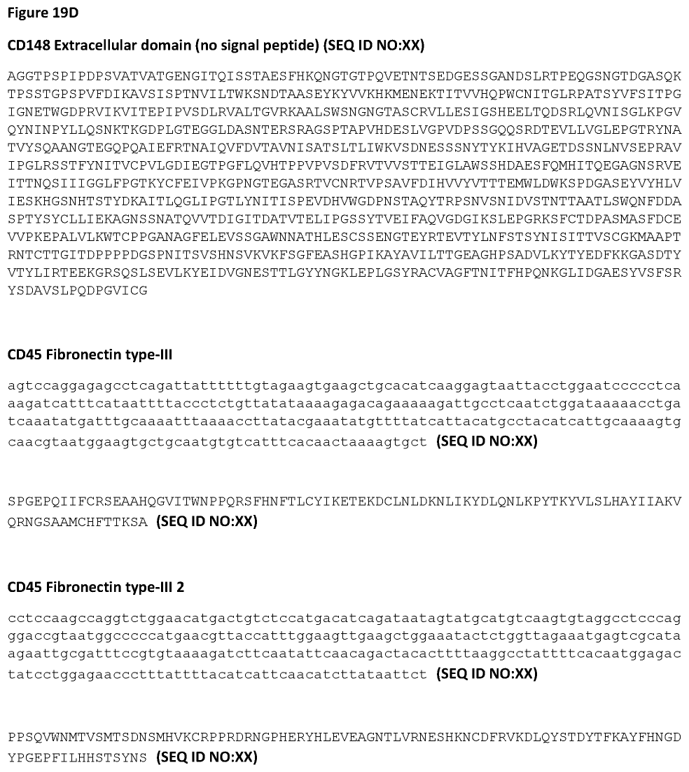

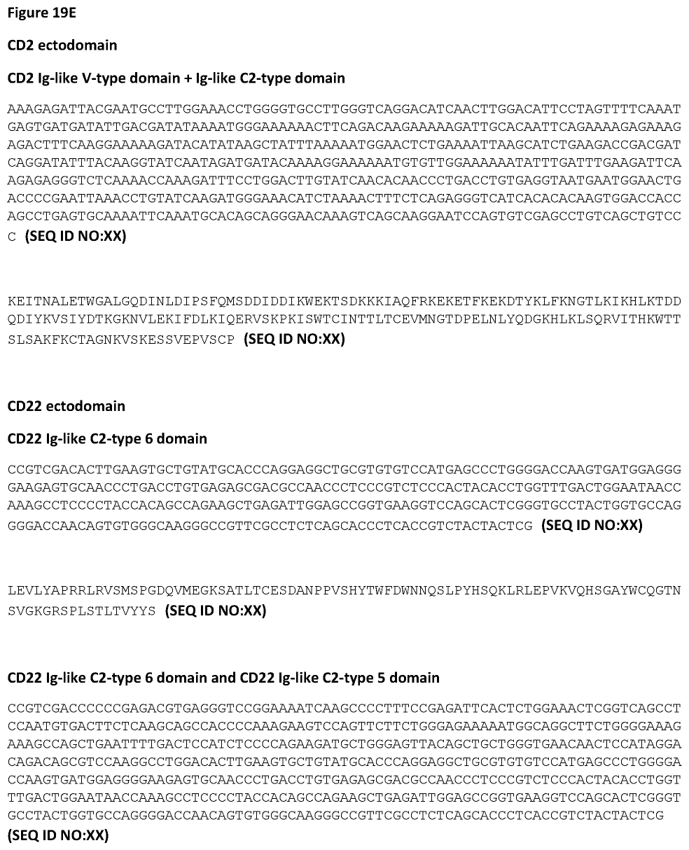

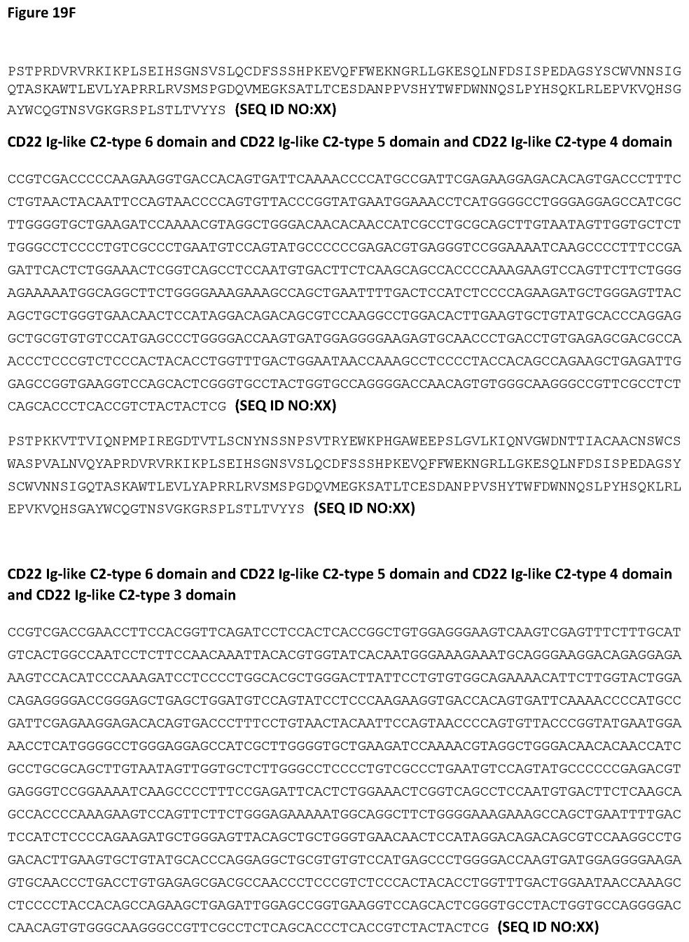

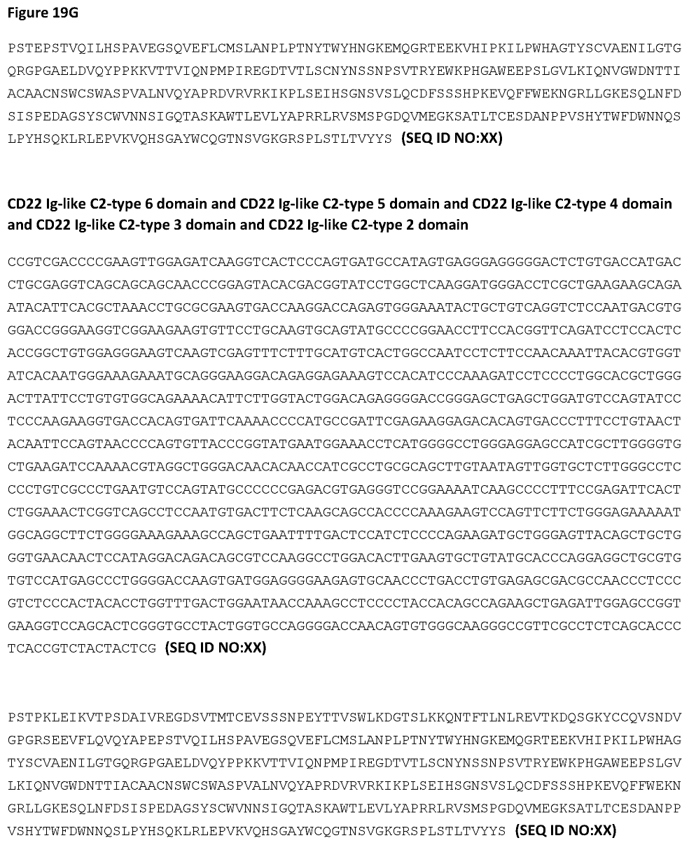

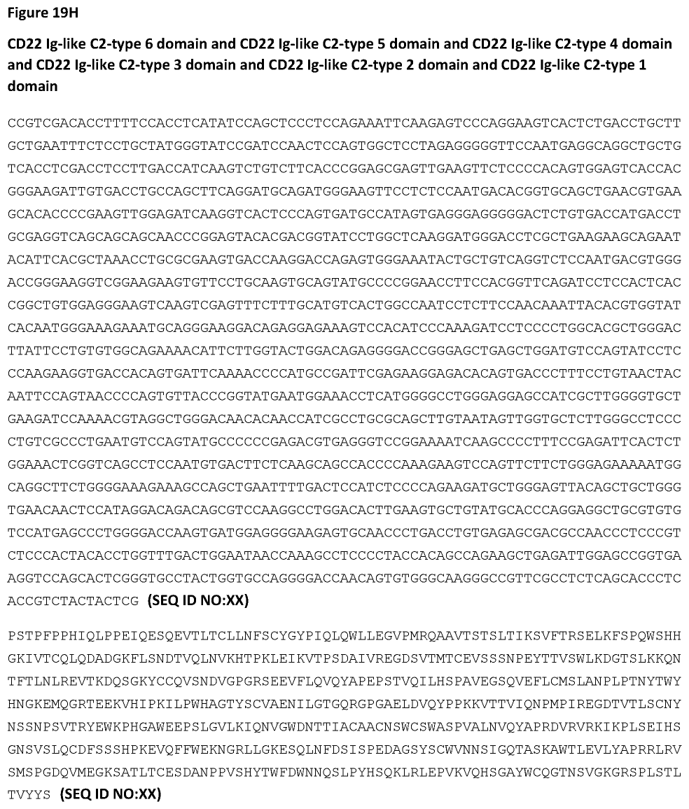

The extracellular domain of the bCAR further includes a rigid elongation domain that is sufficiently rigid to enlarge the immune cell/target cell intermembrane contact zone, thereby inhibiting aCAR/antigen complex and/or TCR/antigen complex formation. See, e.g., FIG. 19.

In certain embodiments, the rigid elongation domain comprises at least one rigid protein module, such as a fibronectin type III repeat or an Ig domain that includes the motifs of the Ig fold. See, e.g., FIG. 19. The Ig domain is a type of protein domain that consists of a 2-layer sandwich of 7-9 antiparallel β-strands arranged in two β-sheets with a Greek key topology, consisting of about 125 amino acids. The backbone switches repeatedly between the two β-sheets., the pattern is (N-terminal β-hairpin in sheet 1)-(β-hairpin in sheet 2)-(β-strand in sheet 1)-(C-terminal β-hairpin in sheet 2). The cross-overs between sheets form an “X”, so that the N- and C-terminal hairpins are facing each other. Members of the immunoglobulin superfamily are found in hundreds of proteins of different functions. Examples include antibodies, the giant muscle kinase titin, and receptor tyrosine kinases. Immunoglobulin-like domains may be involved in protein-protein and protein-ligand interactions.

Fibronectin type-III (FN3) repeats are both the largest and the most common of the fibronectin subdomains. Domains homologous to FN3 repeats have been found in various animal protein families including other extracellular-matrix molecules, cell-surface receptors, enzymes, and muscle proteins. Structures of individual FN3 domains have revealed a conserved beta sandwich fold with one beta sheet containing four strands and the other sheet containing three strands. This fold is topologically very similar to that of Ig-like domains, with a notable difference being the lack of a conserved disulfide bond in FN3 domains.

In some embodiments, the elongation domain of the bCAR includes 1, 2, 3, 4, 5, 6, 7, 8, 9, 10, 11, 12, 13, 14, 15, 16, 17, 18, 19, or 20 rigid protein modules.

In certain embodiments, the elongation domain of the bCAR includes a rigid protein module that is an IgG domain. See, e.g., FIG. 19. In exemplary embodiments, the IgG domain(s) of the rigid protein module is a CD22 and/or CD2 IgG domain. In certain embodiments, the elongation domain comprises 1, 2, 3, 4, 5, 6, 7, 8, 9, 10, 11, 12, 13, 14, 15, 16, 17, 18, 19, or 20 CD2 IgG domains. In some embodiments, the elongation domain comprises 1, 2, 3, 4, 5, 6, 7, 8, 9, 10, 11, 12, 13, 14, 15, 16, 17, 18, 19, or 20 CD22 IgG domains. In particular embodiments, the elongation domain comprises a combination of CD2 and CD22 IgG domains, wherein the elongation domain includes 2, 3, 4, 5, 6, 7, 8, 9, 10, 11, 12, 13, 14, 15, 16, 17, 18, 19, 20 of the combination of IgG domains. In particular embodiments, the IgG domains are human IgG domains.

In some embodiments, the rigid protein module includes a fibronectin type III repeat. See, e.g., FIG. 19. In some embodiments, the elongation domain comprises 1, 2, 3, 4, 5, 6, 7, 8, 9, 10, 11, 12, 13, 14, 15, 16, 17, 18, 19, or 20 fibronectin type III repeats. In exemplary embodiments, the rigid elongation domain includes 2-3 fibronectin type III repeats. In certain embodiments, the rigid elongation domain essentially consists of the extracellular domain of CD45 or CD148 or a fragment thereof (comprising e.g. two or three fibronectin type III repeats).

In some embodiments, the rigid elongation domain includes the extracellular domain of a CD45. See, e.g., FIG. 19. The CD45 family of transmembrane protein tyrosine phosphatases plays a critical role in lymphocyte activation by regulating the phosphorylation and activity of Src-family protein tyrosine kinases and their substrates. See, e.g., Trowbridge et al., Annu Rev Immunol 12:85-116 (1994). Multiple CD45 isoforms are generated by the alternative splicing of exons 4-6, commonly known as exons A, B, and C. The alternative exons give rise to isoforms that differ in the size of their extracellular domains but share identical cytoplasmic protein tyrosine phosphatase domains. The various isoforms range in size from 180 to 220 kDa, depending on which exons are used. Six different human isoforms of CD45 mRNAs have been isolated, which contain all three exons (ABC isoform), two of the three exons (AB and BC isoform), only one exon (A isoform and B isoform), or no exons (0 isoform) (Hermiston et al. 2003). All of the isoforms have the same eight amino acids at their amino-terminus, which are followed by the various combinations of A, B, and C peptides (66, 47, and 48 amino acids long, respectively). The remaining regions (the 383-amino-acid extracellular region, the 22-amino acid transmembrane peptide, and the 707 amino acid-cytoplasmic region) have the identical sequences in all isoforms. In some embodiments, the rigid elongation domain includes exon A of CD45. In some embodiments, the rigid elongation domain includes exon B of CD45. In some embodiments, the rigid elongation domain includes exon C of CD45. In particular embodiments, the elongation domain includes exons A and B of CD45. In certain embodiments, the elongation domain includes exons B and C of CD45. In certain embodiments, the elongation domain includes exons A and C of CD45. In certain embodiments, the elongation domain includes exons A, B, and C of CD45.

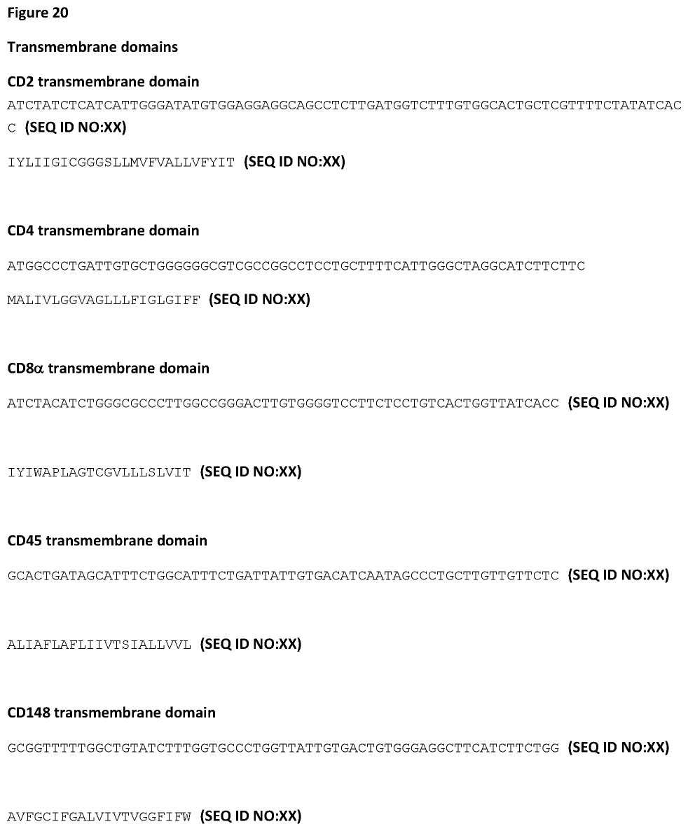

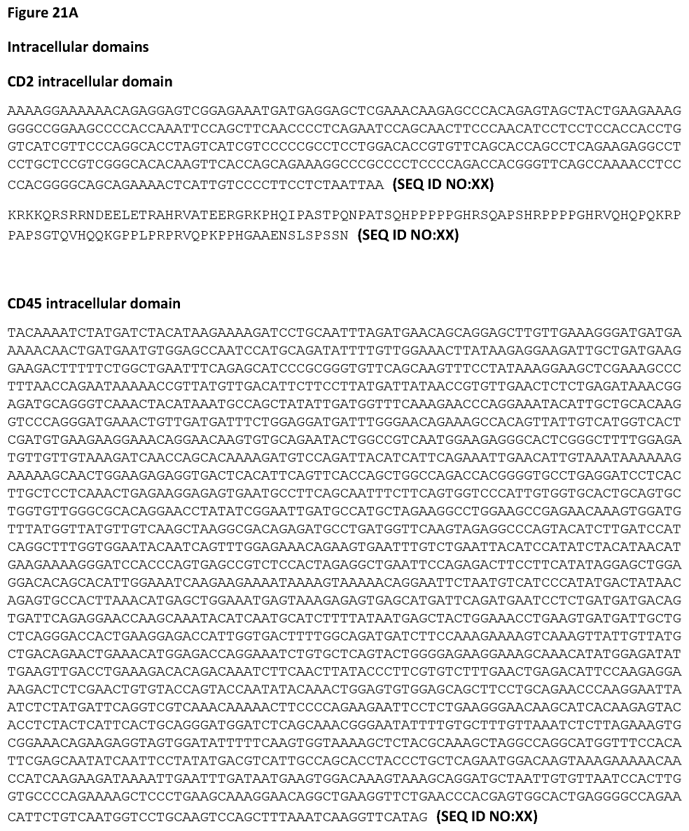

In some embodiments, the elongation domain of the bCAR is linked to the extracellular or transmembrane domain of CD2, CD4, or CD8, or a fragment thereof. See, e.g., FIGS. 19 and 20. In exemplary embodiments, the elongation domain of the bCAR is linked to a CD2, CD4 or CD8 extracellular domain or fragment thereof. In some embodiments, the elongation domain is linked to a CD2, CD4 or CD8 extracellular domain or fragment thereof. In some embodiments, the elongation domain is linked to a CD2, CD4 or CD8 transmembrane domain or fragment thereof. In some embodiments, the elongation domain is linked to the extracellular and transmembrane domain of CD2, CD4 or CD8 or fragment thereof. In certain embodiments, the elongation domain is linked to the extracellular and/or transmembrane domain of CD2 or fragment thereof.

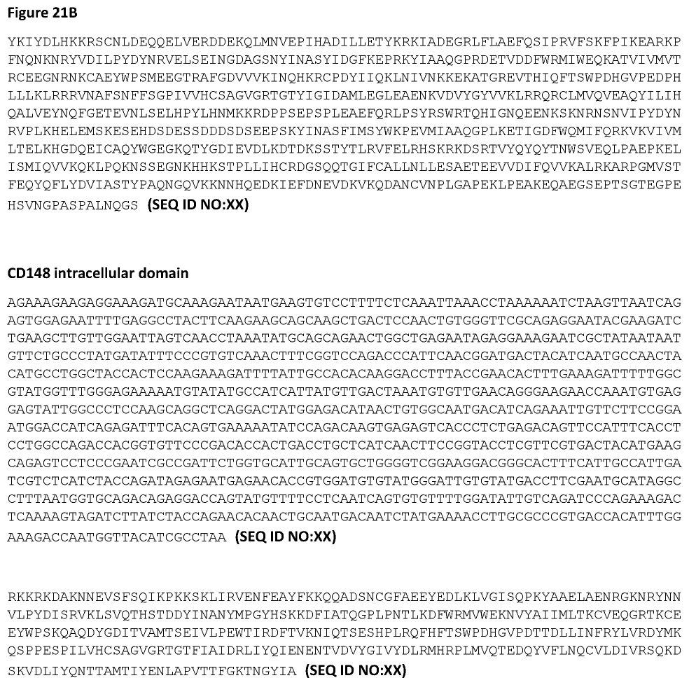

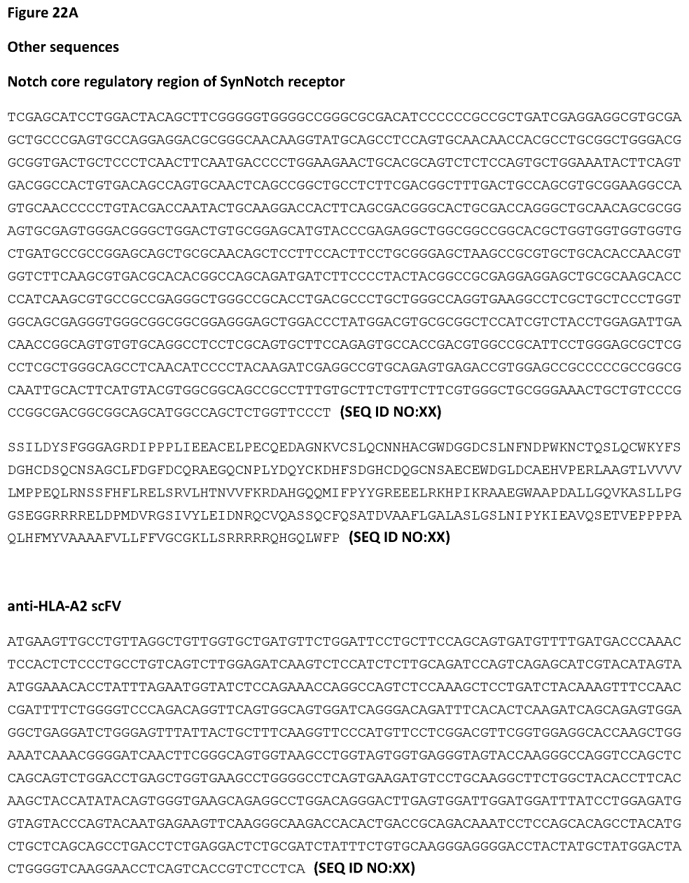

In some embodiments, the extracellular domain of the bCAR is linked to the intracellular domain of CD2, CD45 or CD148 via the transmembrane domain. See, e.g., FIGS. 20 and 21.

In some embodiments, the bCAR includes a CD45 intracellular domain or fragment thereof (i.e., a “CD45-CAR”). See, e.g., FIG. 21. In exemplary embodiments that include such a CD45 intracellular domain, the CD45 intracellular domain exhibits phosphatase activity and is capable of desphorylating phosphorylated ITAMS. Upon binding to its specific antigen (e.g., a cell surface molecule expressed on a target cell to be protected), the elongated bCAR is capable of dephosphorylating phosphorylated ITAMs in the contact zone resulting from antigen binding by the TCR or the aCAR. Thus, the intracellular CD45 domain prevents and/or removes undesired activation cause by aCAR/antigen or TCR/antigen binding.

In some embodiments, the bCAR includes a CD45 extracellular domain or fragment thereof, a CD45 transmembrane domain or fragment thereof, and a CD45 intracellular domain or fragment thereof.

In some embodiments, the bCAR includes a CD2 intracellular domain or fragment thereof (i.e., a “CD2-CAR”). See, e.g., FIG. 21. In exemplary embodiments, the CD2 intracellular domain is capable of association or colocalization with the TCR-CD3 complex. It is believe that the firm cytoskeleton-mediated association between CD2 of a CD2-CAR and the TCR-CD3 complex can effectively counteract opposing segregation forces. While not being bound by theory, it is accordingly believed that in its bound form, the CD2-CAR would remain associated with a coexpressed aCAR and prevent activation signaling of the aCAR. In contrast, upon engagement with target tumor cells that express antigen recognized by the aCAR and do not express antigen recognized by the CD2-CAR, the non-bound CD2-CAR would be excluded from the contact zone, allowing full T cell activation. The CD2-CAR is also expected to prevent the exclusion of native CD45 from the contact zone, retaining its local inhibitory phosphatase activity.

In some embodiments, the intracellular CD2 domain includes one or more mutations that inhibit CD2 co-stimulatory activity. In some embodiments, the mutations are in a PPPGHR sequence in the intracellular domain of CD2. The PPGHR sequence is crucial for binding of an intracellular protein termed CD2 binding protein 2 (CD2BP2). In certain embodiments, the variant CD2 intracellular domain includes a mutation in the binding site for CD2-adaptor protein (CD2AP). Without being bound by any particular theory of operation, it is believed that mutations at these sites can inhibit CD2 costimulatory signaling.

In some embodiments, the bCAR includes an extracellular domain that includes a plurality of CD2 IgG domains, a CD2 transmembrane domain or fragment thereof, and a CD2 intracellular domain or fragment thereof.

In some embodiments, the elongated bCAR includes a binding domain that is an scFv, and an elongation domain that includes a plurality of fibronectin repeats. In some embodiments, the fibronectin repeats are fibronectin type III repeats. In some embodiments, the elongation domain includes a CD45 or CD148 extracellular domain or fragment thereof. In some embodiments, the elongation domain is linked to an extracellular domain and/or transmembrane domain or fragment thereof of CD2, CD4 or CD8. In exemplary embodiments, the elongation domain is linked to an extracellular domain of CD2 or fragment thereof (e.g., a CD2 IgG domain). In exemplary embodiments, the elongation domain is linked to a CD2 transmembrane domain or fragment thereof. In exemplary embodiments, the elongation domain is linked to the transmembrane domain of CD2. In particular embodiments, the transmembrane domain is further linked to intracellular domain of CD45, CD148 or CD2 or a fragment thereof. In some embodiments, the elongation domain is linked to the transmembrane and intracellular domain of CD2.

In some embodiments, the elongated bCAR includes a binding domain that is an scFv, and an elongation domain that includes a plurality of fibronectin repeats. In some embodiments, the fibronectin repeats are fibronectin type III repeats. In some embodiments, the elongation domain is linked to an extracellular domain and/or transmembrane domain or fragment thereof of CD2. In exemplary embodiments, the elongation domain is linked to the transmembrane domain of CD2. In particular embodiments, the transmembrane domain is further linked to intracellular domain of CD2 or a fragment thereof. In some embodiments, the elongation domain is linked to the transmembrane and intracellular domain of CD2.

In some embodiments, the elongated bCAR includes a binding domain that is an scFv, and an elongation domain that includes a plurality of IgG domains. In some embodiments, the IgG domains are CD2 IgG domains, CD22 IgG domains or a combination thereof. In some embodiments, the elongation domain is linked to an extracellular domain and/or transmembrane domain or fragment thereof of CD2. In exemplary embodiments, the elongation domain is linked to the transmembrane domain of CD2. In particular embodiments, the transmembrane domain is further linked to intracellular domain of CD2 or a fragment thereof. In some embodiments, the elongation domain is linked to the transmembrane and intracellular domain of CD2.

In some embodiments, an elongated bCARs that binds a first antigen is co-expressed in an immune cell together with an activating CAR (aCAR) that binds a second antigen. The bCAR allows activation of the immune cell via the aCAR in cells that express the second antigen, but do not express the first antigen.

In some embodiments, the bCAR prevents the undesired activation of an immune cell caused by specific binding of the immune cell to normal tissue via one or more cell-surface epitopes present on normal and related abnormal mammalian tissue. In some embodiments, the immune cell includes an elongated bCAR that binds to a first cell surface antigen present on normal tissue, but absent on related abnormal mammalian tissue. In such embodiments, the elongated bCAR inhibits activation by normal tissue that express the first cell surface antigen. In certain embodiments, the immune cell also expresses an aCAR that binds to a second antigen that is present on a tumor cell. In such embodiments, the immune cell is selectively activated by tumor cells that express the second antigen and exhibits limited activation by normal tissue due to inhibition by the bCAR. In some embodiments, the immune cell is a T cell. In exemplary embodiments, the T cell is an effector T cell. In certain embodiments, the effector immune cell, is an effector T cell; or a regulatory immune cell, such as a regulatory T cell or a subset of natural killer T cells (iNKT cells) and of γδ T cells.

In particular embodiments, the abnormal tissue is a pre-malignant tissue or a malignant tumor tissue. In some embodiments, the bCAR binds to a single allelic variant of a polymorphic cell surface epitope not expressed by the abnormal mammalian tissue but present on all cells of related mammalian normal tissue. In some embodiments, the cell surface epitope is a cell surface epitope of an essential tissue-associated gene product or housekeeping gene products. In exemplary embodiments, the cell surface epitope is an ion channel or a receptor tyrosine kinase.

In other embodiments, the bCAR prevents the undesired activation of an immune cell caused by specific binding of the immune cell to normal tissue via one or more cell-surface epitopes present on normal and mammalian tissue being targeted by an autoimmune response. In some embodiments, the immune cell includes an elongated bCAR that specifically binds to a first antigen (e.g., a cell surface antigen) present on normal mammalian tissue, but absent on mammalian tissue being targeted by an autoimmune response. In exemplary embodiments, the immune cell is a regulatory immune cell (e.g., a Treg cell) that is capable of activation by a second antigen (e.g., a second cell surface antigen) present on mammalian tissue being targeted by the autoimmune response. Thus in such embodiments, the regulatory immune cell is selectively activated by tissue that express the second antigen and exhibits limited activation by normal tissue due to inhibition by the bCAR. In some embodiments, the immune cell (e.g., Treg cell) suppresses the autoimmune response upon activation. Exemplary mammalian tissue being targeted by the autoimmune response include, but is not limited to, pancreatic islets of the pancreas tissue; digestive system tissue; small intestine tissue; large intestine tissue; colon; thyroid tissue; nervous system tissue; skin tissue, thyroid tissue, or articular joints.

B. CD45N-CARs

Energy-wise, segregation of a bCAR from the contact zone following antigen binding would be less favorable than that of its non-bound counterpart owing to anchorage of the antigen at the target cell membrane and/or to the TCR-CD3 complex, resisting lateral diffusion. Nonetheless, one cannot rule out the possibility that even upon antigen-binding, CD45-CAR can still be excluded from the contact zone, albeit less efficiently than native CD45. In some embodiments, the bCAR as described provides for a conditionally cleavable bCAR that will retain membrane attachment.

In order to provide for an AND NOT gate that is not dependent on calibration of the catalytic rate of the endodomain phosphatase such that significant dephosphorylation of ITAMS only occurs when activating and inhibitory endodomains are co-localized, an alternative genetic device is provided herein that is designed to redirect the inhibitory phosphatase activity of a powerful phosphatase, such as CD45, to the contact zone upon antigen binding by the bCAR, harnessing regulated intramembrane proteolysis (RIP). RIP is a fundamental cellular process that directs the cleavage of integral cell surface receptors by distinct proteolytic enzymes. Ligand binding by RIP-controlled receptors sequentially liberates their extracellular and intracellular domains to execute diverse regulatory functions. RIP has been recruited to create genetic switches for controlled combinatorial CAR-mediated recognition of target cells, applying the Notch receptor (See, e.g., Roybal et al. Cell 167:419-432 (2016); Morsut et al., Cell 164:780-791; and Roybal et al., Cell 164:770-779 (2016)). In these studies, binding of the RIP-based CAR to one antigen releases a genetically-engineered intracellular domain incorporating an exogenous transcription factor of microbial origin that translocates to the cell nucleus where it turns on the expression of the second CAR by binding to its unique promoter. The current application utilizes this process solely for removing the entire bCAR ectodomain upon antigen binding, leaving the transmembrane and the intracellular phosphatase portion intact (FIG. 5E). This “minimal” phosphatase moiety, termed CD45N-CAR, would not be excluded from the contact zone and, consequently, inhibit T cell activation, as has been generally for genetically truncated CD45 and CD148. Irles et al., Nat. Immunol. 4:189-197 (2003); and Cordoba et al., Blood 121:4295-302 (2013).

In another aspect, provided herein is a blocking CAR termed “CD45N-CAR” (FIGS. 5A and E). Such CD45N-CARs include an extracellular domain that includes a single-chain binding domain, a rigid elongation domain, and a protease cleavage site that is capable of being recognized and cleaved by a protease (e.g., a sheddase). The extracellular domain of the CD45N-CAR is linked to an intracellular domain by a transmembrane domain. The intracellular domain includes a full or partial intracellular domain of a membrane phosphatase capable of dephosphorylating phosphorylated immunoreceptor tyrosine-based activation motifs (ITAMs).

As shown in FIG. 5E, immune cells that express CD45N-CAR undergo protease mediated cleavage of the extracellular domain upon binding to antigen in the contact zone. Cleavage of the CD45N-CAR, results in a cleaved extracellular domain that is excluded from the contact zone and a membrane-bound transmembrane domain-intracellular domain portion that is not excluded from the contact zone. The transmembrane domain-intracellular domain portion of the CD45N-CAR consequently inhibits immune cell activation by dephosphorylating ITAMs of activated aCARs or TCRs in the contact zone vicinity. Thus, the CD45N-CAR allows for activation of a co-expressed aCAR or TCR only in the presence of the specific antigen for the aCAR or TCR and in the absence of a “forbidden” antigen recognized by the CD45N-CAR.

In certain embodiments, the length of the extracellular domain of the CD45N-CAR is more than 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 nm in length. In exemplary embodiments, the extracellular domain is more than about 20 nm in length.

The protease cleavage site of the CD45N-CAR is positioned in a manner such that cleavage of the site in a CD45N-CAR-expressing immune cell results in an intact transmembrane domain-intracellular domain portion that is membrane bound in the contact zone. The resulting transmembrane domain-intracellular domain portion of the CD45N-CAR is capable of inhibiting immune cell activation by dephosphorylating ITAMs of activated aCARs or TCRs in the contact zone vicinity.

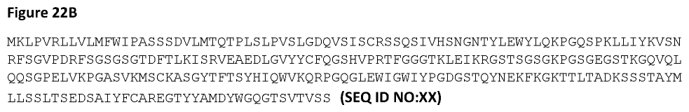

In certain embodiments, the protease cleavage site includes a Notch core regulatory region of a synthetic Notch (SynNotch) receptor or variant thereof. See, e.g., FIG. 22. See, e.g., Roybal et al. Cell 167:419-432 (2016) and Roybal et al., Cell 164:770-779 (2016), which are incorporated by reference in pertinent parts relating to SynNotch receptors and Notch core regulatory regions. Such a core regulatory region allows for cleavage of the protease cleavage site upon ligand binding of the CD45N-CAR. In some embodiments, the protease cleavage site includes Lin 12/Notch repeats and/or an ADAM protease cleavage site. In certain embodiments, the protease is a disintegrin and metalloproteinase (ADAM) or a beta-secretase 1 (BACE1).

Any suitable single chain binding domain can be included in the extracellular domain that allows for binding to an epitope of a target cell, including the exemplary single chain binding domains described herein (see, Section III.A). In some embodiments, the binding domain is a single chain variable fragment.

In exemplary embodiments, the extracellular domain of the CD45N-CAR includes at least one rigid protein module, such as a fibronectin type III repeat or an Ig domain that includes the motifs of the Ig fold. See, e.g., FIG. 19. In some embodiments, the elongation domain of the CD45N-CAR includes 1, 2, 3, 4, 5, 6, 7, 8, 9, 10, 11, 12, 13, 14, 15, 16, 17, 18, 19, or 20 rigid protein modules.

In certain embodiments, the elongation domain of the CD45N-CAR includes a rigid protein module that is an IgG domain. See, e.g., FIG. 19. In exemplary embodiments, the IgG domain(s) of the rigid protein module is a CD22 and/or CD2 IgG domain. In certain embodiments, the elongation domain comprises 1, 2, 3, 4, 5, 6, 7, 8, 9, 10, 11, 12, 13, 14, 15, 16, 17, 18, 19, or 20 CD2 IgG domains. In some embodiments, the elongation domain comprises 1, 2, 3, 4, 5, 6, 7, 8, 9, 10, 11, 12, 13, 14, 15, 16, 17, 18, 19, or 20 CD22 IgG domains. In particular embodiments, the elongation domain comprises a combination of CD2 and CD22 IgG domains, wherein the elongation domain includes 2, 3, 4, 5, 6, 7, 8, 9, 10, 11, 12, 13, 14, 15, 16, 17, 18, 19, 20 of the combination of IgG domains. In particular embodiments, the IgG domains are human IgG domains.

In some embodiments, the rigid protein module includes a fibronectin type III repeat. See, e.g., FIG. 19. In some embodiments, the elongation domain comprises 1, 2, 3, 4, 5, 6, 7, 8, 9, 10, 11, 12, 13, 14, 15, 16, 17, 18, 19, or 20 fibronectin type III repeats. In exemplary embodiments, the rigid elongation domain includes 2-3 fibronectin type III repeats. In certain embodiments, the rigid elongation domain essentially consists of the extracellular domain of CD45 or CD148 or a fragment thereof (comprising e.g. two or three fibronectin type III repeats).

In some embodiments, the rigid elongation domain includes the extracellular domain of a CD45 as discussed herein. See, e.g., FIG. 19. In some embodiments, the rigid elongation domain includes exon B of CD45. In some embodiments, the rigid elongation domain includes exon C of CD45. In particular embodiments, the elongation domain includes exons A and B of CD45. In certain embodiments, the elongation domain includes exons B and C of CD45. In certain embodiments, the elongation domain includes exons A and C of CD45. In certain embodiments, the elongation domain includes exons A, B, and C of CD45.

In some embodiments, the extracellular domain of the CD45N-CAR is linked to the intracellular domain via the transmembrane domain. In some embodiments, the protease cleavage site of the extracellular domain is linked to intracellular domain via the transmembrane domain. Cleavage of the protease cleavage site results in an intact transmembrane-intracellular domain portion.

The intracellular domain of the CD45N-CAR includes partial intracellular domain of a membrane phosphatase capable of dephosphorylating phosphorylated immunoreceptor tyrosine-based activation motifs (ITAMs). In exemplary embodiments, the intracellular domain of the CD45N-CAR is a CD45 intracellular domain, a CD148 intracellular domain, or fragment thereof. In exemplary embodiments that include such a CD45 intracellular domain, the CD45 intracellular domain exhibits phosphatase activity and is capable of desphorylating phosphorylated ITAMS. Upon binding to its specific antigen (e.g., a cell surface molecule expressed on a target cell to be protected), the CD45N-CAR undergoes protease cleavage that results in an intact transmembrane-intracellular domain portion that is present in the contact zone. The intact transmembrane-intracellular domain portion is capable of dephosphorylating any phosphorylated ITAMs in the contact zone resulting from antigen binding by the TCR or the aCAR. Thus, the intact transmembrane-intracellular domain portion prevents and/or removes undesired activation cause by aCAR/antigen or TCR/antigen binding.

In some embodiments, the CD45N-CAR includes a binding domain that is an scFv, and an elongation domain that includes a plurality of fibronectin repeats. In some embodiments, the fibronectin repeats are fibronectin type III repeats. In some embodiments, the elongation domain includes a CD45 or CD148 extracellular domain or fragment thereof. In some embodiments, the elongation domain is linked to an extracellular domain and/or transmembrane domain or fragment thereof of CD2, CD4, CD8 or CD45. In particular embodiments, the transmembrane domain is further linked to intracellular domain of CD45 or a fragment thereof.

In some embodiments, the CD45N-CAR includes a binding domain that is an scFv and a protease cleavage site that is capable of being cleaved by a disintegrin and metalloproteinase (ADAM) or a beta-secretase 1 (BACE1).

In some embodiments, the CD45N-CAR includes a binding domain that is an scFv, and an elongation domain that includes a plurality of fibronectin repeats. In some embodiments, the fibronectin repeats are fibronectin type III repeats. In some embodiments, the elongation domain includes a CD45 or CD148 extracellular domain or fragment thereof. In some embodiments, the CD45N-CAR includes a protease cleavage site that is capable of being cleaved by a disintegrin and metalloproteinase (ADAM) or a beta-secretase 1 (BACE1).

In some embodiments, the elongated CD45N-CAR includes a binding domain that is an scFv and an elongation domain that includes a plurality of fibronectin repeats. In some embodiments, the fibronectin repeats are fibronectin type III repeats. In some embodiments, the elongation domain includes a CD45 or CD148 extracellular domain or fragment thereof. In some embodiments, the elongation domain is linked to an extracellular domain and/or transmembrane domain or fragment thereof of CD2. In particular embodiments, the transmembrane domain is further linked to intracellular domain of CD45, CD148 or CD2 or a fragment thereof.

In some embodiments, the elongated CD45N-CAR includes a binding domain that is an scFv and an elongation domain that includes a plurality of fibronectin repeats. In some embodiments, the fibronectin repeats are fibronectin type III repeats. In some embodiments, the elongation domain includes a CD45 or CD148 extracellular domain or fragment thereof. In some embodiments, the CD45N-CAR includes a protease cleavage site that is capable of being cleaved by a disintegrin and metalloproteinase (ADAM) or a beta-secretase 1 (BACE1).

In particular embodiments, the transmembrane domain is further linked to the intracellular domain of CD45 or CD148 or a fragment thereof. See, e.g., FIG. 21.

In some embodiments, the elongated CD45N-CAR includes a binding domain that is an scFv, and an elongation domain that includes a plurality of IgG domains. In some embodiments, the IgG domains are CD2 IgG domains, CD22 IgG domains or a combination thereof. In some embodiments, the elongation domain is linked to an extracellular domain and/or transmembrane domain or fragment thereof of CD2 or CD45. In exemplary embodiments, the elongation domain is linked to the transmembrane domain of CD2. In particular embodiments, the transmembrane domain is further linked to intracellular domain of CD45 or CD148 or a fragment thereof.

In some embodiments, a CD45N-CAR that binds a first antigen is co-expressed in an immune cell together with an activating CAR (aCAR) that binds a second antigen. The CD45N-CAR allows activation of the immune cell via the aCAR upon contact with cells that express the second antigen, but do not express the first antigen.

In some embodiments, the CD45N-CAR prevents the undesired activation of an immune cell caused by specific binding of the immune cell to normal tissue via one or more cell-surface epitopes present on normal and related abnormal mammalian tissue. In some embodiments, the immune cell includes a CD45N-CAR that binds to a first cell surface antigen present on normal tissue, but absent on related abnormal mammalian tissue. In such embodiments, the elongated CD45N-CAR inhibits activation by normal tissue that express the first cell surface antigen. In certain embodiments, the immune cell also expresses an aCAR that binds to a second antigen that is present on a tumor cell. In such embodiments, the immune cell is selectively activated by tumor cells that express the second antigen and exhibits limited activation by normal tissue due to inhibition by the CD45N-CAR. In some embodiments, the immune cell is a T cell. In exemplary embodiments, the T cell is an effector T cell. In certain embodiments, the effector immune cell, is an effector T cell; or a regulatory immune cell, such as a regulatory T cell or a subset of natural killer T cells (iNKT cells) and of γδ T cells.

In particular embodiments, the abnormal tissue is a pre-malignant tissue or a malignant tumor tissue. In some embodiments, the CD45N-CAR binds to a single allelic variant of a polymorphic cell surface epitope not expressed by the abnormal mammalian tissue but present on all cells of related mammalian normal tissue. In some embodiments, the cell surface epitope is a cell surface epitope of an essential tissue-associated gene product or housekeeping gene products. In exemplary embodiments, the cell surface epitope is an ion channel or a receptor tyrosine kinase.

In other embodiments, the CD45N-CAR prevents the undesired activation of an immune cell caused by specific binding of the immune cell to normal tissue via one or more cell-surface epitopes present on normal and mammalian tissue being targeted by an autoimmune response. In some embodiments, the immune cell includes an CD45N-CAR that specifically binds to a first antigen (e.g., a cell surface antigen) present on normal mammalian tissue, but absent on mammalian tissue being targeted by an autoimmune response. In exemplary embodiments, the immune cell is a regulatory immune cell (e.g., a Treg cell) that is capable of activation by a second antigen (e.g., a second cell surface antigen) present on mammalian tissue being targeted by the autoimmune response. Thus in such embodiments, the regulatory immune cell is selectively activated by tissue that express the second antigen and exhibits limited activation by normal tissue due to inhibition by the CD45N-CAR. In some embodiments, the immune cell (e.g., Treg cell) suppresses the autoimmune response upon activation. Exemplary mammalian tissue being targeted by the autoimmune response include, but is not limited to, pancreatic islets of the pancreas tissue; digestive system tissue; small intestine tissue; large intestine tissue; colon; thyroid tissue; nervous system tissue; skin tissue, thyroid tissue, or articular joints.

C. sCD45-CARs

The potential exclusion of antigen-bound CD45-CAR from the contact zone puts forward a unique solution to the challenge of combinatorial antigen recognition (AND gate), taking advantage of the existence of an exclusion threshold below which no effective exclusion takes place. In some embodiments, the bCAR as described provides for a conditionally cleavable bCAR that will retain membrane attachment.

In another aspect, provided herein is a blocking CAR termed “sCD45-CAR”. The sCD45-CAR is a shortened version of the CD45-CAR described herein and includes an extracellular domain includes a single-chain binding domain and an elongation domain. The sCD45-CAR further includes an intracellular domain that is attached to the extracellular domain by a transmembrane domain. The intracellular domain includes a full or partial intracellular domain of a membrane phosphatase capable of dephosphorylating phosphorylated immunoreceptor tyrosine-based activation motifs (ITAMs).

A key feature of the sCD45-CAR is that it is not excluded from a contact zone in its unbound state (FIG. 5F, left). Consequently, the unbound sCD45-CAR is able to desphosporylate ITAMs and prevent T cell activation from neighboring aCAR/antigen binding in the contact zone (FIG. 5F, left). In contrast, antigen-binding of sCD45-CAR results in a larger molecular complex which exceeds the exclusion threshold. The enforced segregation of sCD45-CAR/antigen from the contact zone abolishes the local inhibitory phosphatase activity of the sCD45-CAR, leading to TCR- or aCAR-mediated T cell activation (FIG. 5F, right).

In some embodiments, the length or size of the extracellular domain of the sCD45-CAR is approximately equal to that of the extracellular domain of a TCR or aCAR. In particular embodiments, the extracellular domain is less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 nm in length. In exemplary embodiments, the extracellular domain is less than 20 nm in length. As the sCD45-CAR is excluded from the contact zone upon binding to its target antigen, the sCD45-CAR binds to antigens on target cells that would create distances larger that the contact zone exclusion threshold upon sCD45-CAR/antigen binding. In particular embodiments, the sCD45-CAR binds to a membrane-distal epitopes on a target cells that creates this contact zone exclusion threshold effect. In exemplary embodiments the total length of the sCD45-CAR extracellular domain and bound cell surface epitope of the target cell is more than 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50 nm. In particular embodiments, the total length of the sCD45-CAR extracellular domain and bound cell surface epitope of the target cell is more than 20 nm. While other blocking CARs provided herein prevent T cell reactivity in the presence of antigen and exert an inhibitory effect, sCD45-CAR permit T cell reactivity only in the presence of the antigen, thereby fulfilling a licensing role.

Any suitable single chain binding domain can be included in the extracellular domain that allows for binding to an epitope of a target cell, including the exemplary single chain binding domains described herein (See Section III.A). In some embodiments, the binding domain is a single chain variable fragment.

In some embodiments, the extracellular domain of the sCD45-CARs includes at least one rigid protein module, such as a fibronectin type III repeat or an Ig domain that includes the motifs of the Ig fold. See, e.g., FIG. 19. In some embodiments, the elongation domain of the CD45N-CAR includes 1, 2, 3, 4, 5, 6, 7, 8, 9, or 10 rigid protein modules.

In certain embodiments, the elongation domain of the sCD45-CAR includes a rigid protein module that is an IgG domain. See, e.g., FIG. 19. In exemplary embodiments, the IgG domain(s) of the rigid protein module is a CD22 and/or CD2 IgG domain. In certain embodiments, the elongation domain comprises 1, 2, 3, 4, 5, 6, 7, 8, 9, or 10 CD2 IgG domains. In some embodiments, the elongation domain comprises 1, 2, 3, 4, 5, 6, 7, 8, 9, or 10 CD22 IgG domains. In particular embodiments, the elongation domain comprises a combination of CD2 and CD22 IgG domains, wherein the elongation domain includes 1, 2, 3, 4, 5, 6, 7, 8, 9, or 10 of the combination of IgG domains. In particular embodiments, the IgG domains are human IgG domains.

In some embodiments, the rigid protein module includes a fibronectin type III repeat. In some embodiments, the elongation domain comprises 1, 2, 3, 4, 5, 6, 7, 8, 9, or 10 fibronectin type III repeats. In exemplary embodiments, the rigid elongation domain includes 2-3 fibronectin type III repeats. In certain embodiments, the rigid elongation domain essentially consists of the extracellular domain of CD45 or CD148 or a fragment thereof (comprising e.g. two or three fibronectin type III repeats).

In some embodiments, the rigid elongation domain includes the extracellular domain of a CD45 as discussed herein. See, e.g., FIG. 19. In some embodiments, the rigid elongation domain includes exon B of CD45. In some embodiments, the rigid elongation domain includes exon C of CD45. In particular embodiments, the elongation domain includes exons A and B of CD45. In certain embodiments, the elongation domain includes exons B and C of CD45. In certain embodiments, the elongation domain includes exons A and C of CD45. In certain embodiments, the elongation domain includes exons A, B, and C of CD45.

In certain embodiments, the intracellular domain of the sCD45-CARs includes partial intracellular domain of a membrane phosphatase capable of dephosphorylating phosphorylated immunoreceptor tyrosine-based activation motifs (ITAMs). In exemplary embodiments, the intracellular domain of the sCD45-CAR is a CD45 intracellular domain, a CD148 intracellular domain, or fragment thereof. See, e.g., FIG. 21. In exemplary embodiments that include such a CD45 intracellular domain, the CD45 intracellular domain exhibits phosphatase activity and is capable of desphorylating phosphorylated ITAMS. Unbound sCD45-CAR is able to desphosporylate ITAMs and prevent T cell activation from neighboring aCAR/antigen binding in the contact zone (FIG. 5F, left).

In some embodiments, the sCD45-CAR provides a licensing role and therefore binds specifically to a cell surface epitope present on abnormal mammalian tissue (e.g., a tumor), but not on normal mammalian tissue. In some embodiments, the sCD45-CAR binds specifically to a cell surface epitope present on mammalian tissue being targeted by an autoimmune response, but not on normal mammalian tissue.

In some embodiments, an immune cell co-expresses: a) an sCD45-CAR that binds specifically to a first cell surface epitope present abnormal mammalian tissue (e.g., a tumor), but not on normal mammalian tissue; and b) an activating CAR (aCAR) that binds specifically to a second cell surface epitope present on the abnormal mammalian tissue. In particular embodiments, the second cell surface epitope is not present on normal tissue. In some embodiments, the immune cell is a T cell.

In exemplary embodiments, the T cell is an effector T cell. In particular embodiments, the abnormal tissue is a pre-malignant tissue or a malignant tumor tissue. In some embodiments, the bCAR binds to a single allelic variant of a polymorphic cell surface epitope expressed by the abnormal mammalian tissue but not present on all cells of related mammalian normal tissue. In some embodiments, the cell surface epitope is a cell surface epitope of an essential tissue-associated gene product or housekeeping gene products. In exemplary embodiments, the cell surface epitope is an ion channel or a receptor tyrosine kinase.

In certain embodiments, an immune cell (e.g., a regulatory immune cell) co-expresses: a) an sCD45-CAR that binds specifically to a first cell surface epitope present on mammalian tissue being targeted by an autoimmune response, but not on normal mammalian tissue; and b) an activating CAR (aCAR) that binds specifically to a second cell surface epitope present on mammalian tissue being targeted by the autoimmune response. In particular embodiments, the second cell surface epitope is not present on normal tissue.

In some embodiments, a prerequisite for full functionality of sCD45-CARs is that the level of antigen exceeds that of receptor molecules at the T cell surface so that the amount of non-bound, non-excluded receptors remains sufficiently low, thus incapable of inhibiting activation.

D. Nucleic Acid Compositions

In another aspect, provided herein are polynucleotides encoding the blocking CARs. The present invention also provides vectors in which a polynucleotide encoding a subject blocking CAR is inserted. In some embodiments, the vector is derived from a retrovirus. In exemplary embodiments, the retrovirus is a lentivirus. In some embodiments, the retroviral vector is a gammaretroviral vector.

In another embodiment, the vector comprising the nucleic acid encoding the desired blocking CAR is an adenoviral vector. In another embodiment, the expression of polynucleotides encoding CARs can be accomplished using of transposons such as CRISPR, CAS9, and zinc finger nucleases.

In some embodiments, the vector includes a signal sequence to facilitate secretion, a polyadenylation signal and transcription terminator (e.g., from Bovine Growth Hormone (BGH) gene), an element allowing episomal replication and replication in prokaryotes (e.g., SV40 origin and ColE1 or others known in the art) and/or elements to allow selection (e.g., ampicillin resistance gene and/or zeocin marker).

The expression of natural or synthetic nucleic acids encoding the subject blocking CARs is typically achieved by operably linking a nucleic acid encoding the subject blocking CAR or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence. In some embodiments, additional promoter elements (e.g., enhancers) that regulate the frequency of transcriptional initiation are included.

E. Blocking CAR Expressing Cells

In another aspect, provided herein are immune cells that include one or more of the blocking CARs provided herein. In some embodiments the immune cell is an immune effector cell. Immune effector cells include, but are not limited to, alpha/beta T cells and gamma/delta T cells, B cells, natural killer (NK) cells, natural killer T (NK-T) cells, mast cells, and myeloid-derived phagocytes. In certain embodiments, the immune cell is a regulatory immune cell (e.g., a Treg cell).

Immune cells that express the subject blocking CARs provided herein can be made using any suitable technique in the art. Nucleic acid compositions that encode for the blocking CAR may be added to the host immune cell by conjugation, transformation, transfection, electroporation, etc., where the added nucleic acid molecule is incorporated into the host cell genome or may exist as extrachromosomal genetic material (e.g., as a plasmid or other self-replicating vector). Expression of the blocking CAR can be detected using any suitable technique including flow cytometry techniques.

In particular embodiments, the immune cell is obtained prior to genetic modification. Exemplary sources of such immune cells include, but are not limited to, humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

Antigen binding by bCARs would only affect the contact zone with a cell expressing that antigen while full T cell functionality is preserved, allowing the simultaneous response to other cells and maximizing the efficacy of treatment. Collectively, the bCAR concept offers entirely new solutions to the targeting of yet more complex antigenic signatures so as to further increase the safety of CAR T cell therapy. For example, different (≥2) CD45-CARs, CD2-CARs and CD45N-CARs can be simultaneously expressed by the same T cells, limiting T cell activation only to tumor or other target cells exhibiting ≥2 antigenic “absences”. Likewise, the anticipated mode of action of sCD45-CAR would enable redirecting CAR T cells against 3 or more antigens in an utterly selective manner, expanding the range of target cells while minimizing off-target cell toxicities. Furthermore, as all components are of human origin, no host immune response against the therapeutic T cells is expected.

In some embodiments, the immune cell includes 1, 2, 3, 4, 5 or more different blocking CARs. In certain embodiments, the immune cell further comprises 1, 2, 3, 4, 5, 6, 7, 8, 9, 10 or more different activating CARs.

In certain embodiments, the immune cell includes a) a blocking CAR that binds to a first cell surface antigen present on normal tissue, but absent on related abnormal mammalian tissue; and an aCAR that binds to a second antigen that is present the abnormal tissue. In particular embodiments, the immune cell is an immune effector cell. In particular embodiments, the abnormal tissue is a tumor tissue.

In some embodiments, the immune cell includes a blocking CAR that binds to a first antigen (e.g., a cell surface antigen) present on normal mammalian tissue, but absent on mammalian tissue being targeted by an autoimmune response. In exemplary embodiments, the immune cell is a regulatory immune cell (e.g., a Treg cell) that is capable of activation by a second antigen (e.g., a second cell surface antigen) present on mammalian tissue being targeted by the autoimmune response.

In some embodiments, the immune cell includes a blocking CAR that binds specifically to a first cell surface epitope present abnormal mammalian tissue (e.g., a tumor), but not on normal mammalian tissue; and b) an activating CAR (aCAR) that binds specifically to a second cell surface epitope present on the abnormal mammalian tissue. In particular embodiments, the second cell surface epitope is not present on normal tissue. In some embodiments, the immune cell is an immune effector cell.

In certain embodiments, an immune cell includes a blocking CAR that binds specifically to a first cell surface epitope present on mammalian tissue being targeted by an autoimmune response, but not on normal mammalian tissue; and b) an activating CAR (aCAR) that binds specifically to a second cell surface epitope present on mammalian tissue being targeted by the autoimmune response. In particular embodiments, the second cell surface epitope is not present on normal tissue.

F. Pharmaceutical Compositions

In another aspect, provided herein are pharmaceutical compositions that include immune cells expressing a subject blocking CAR together with a pharmaceutically acceptable diluent or carrier. In exemplary embodiments, the pharmaceutical composition is present in a form suitable for intravenous administration.

The pharmaceutical compositions provided herein include various dosage forms. The pharmaceutical composition is preferably administered parenterally, particularly preferably intravenously. In one embodiment of the invention, the parenteral pharmaceutical composition is present in a dosage form suitable for injection. Thus, a particularly preferred composition is a solution, emulsion or suspension of cells and target modules present in a pharmaceutically acceptable diluent or carrier.

The pharmaceutical composition must be sterile and stable under the conditions of manufacture and storage. The composition can be formulated as a solution, microemulsion, dispersion, in liposomes, or other ordered structures suitable for this purpose.

G. Methods of Treatment

In another aspect, provided herein are methods of treatment using immune cells that express the subject blocking CAR and, optionally, an activating CAR (aCAR).

In some embodiments, the subject immune cells (e.g., effector T cell) are used for the treatment of a cellular proliferative disease, such as a cancer, in a human or animal subject in need of such a treatment. In one embodiment, the method comprises administering to the subject a therapeutically effective amount of a pharmaceutical compositions provided herein, either alone or in combination with other anti-cancer agents.

Examples of cancers that can be treated with the subject immune cells include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments of the method, the subject immune cell includes an aCAR that binds to a specific tumor associated antigen (TAA) expressed by the cancer. TAAs include, but are not limited to, B7H, CD20, CD38, CD123; ROR1, ROR2, BCMA; PSMA; SSTR2; SSTR5, CD19, FLT3, CD33, PSCA, ADAM 17, CEA, Her2, EGFR, EGFR-vIII, CD30, FOLR1, GD-2, CA-IX, Trop-2, CD70, CD38, mesothelin, EphA2, CD22, CD79b, GPNMB, CD56, CD138, CD52, CD74, CD30, CD123, RON, ERBB2, and EGFR.

In other embodiments, the subject immune cells are used to suppress an immune response associated with an autoimmune disease. In one embodiment, provided herein is a method of treating human or animal subjects having an autoimmune disease, and in need of such treatment. In some embodiments, the method comprises administering to the subject a therapeutically effective amount of a pharmaceutical compositions provided herein. Exemplary autoimmune diseases include, but are not limited to rheumatoid arthritis, systemic lupus erythematosus, multiple sclerosis, Type 1 diabetes mellitus, chronic inflammatory demyelinating polyneuropathy, psoriasis, Hashimoto's thyroiditis, Myasthenia gravis, and vasculitis.

### EXEMPLARY EMBODIMENTS

1. A nucleic acid molecule comprising a nucleotide sequence encoding a blocking chimeric antigen receptor (bCAR) capable of preventing undesired activation of an immune cell, wherein the bCAR comprises an extracellular domain comprising:

(i) a single-chain binding domain, a rigid elongation domain and a substrate for a sheddase linked via a transmembrane domain to an intracellular domain comprising a full or partial intracellular domain of a membrane phosphatase capable of dephosphorylating phosphorylated immunoreceptor tyrosine-based activation motifs (ITAMs); or

(ii) a single-chain binding domain and a rigid elongation domain linked via a transmembrane domain to an intracellular domain comprising a module capable of association or colocalization with T cell receptor (TCR)-CD3 complex,

wherein the binding domain specifically binds to a cell surface epitope present on normal mammalian tissue but not on related abnormal mammalian tissue or mammalian tissue being targeted by an autoimmune response, and the length of the extracellular domain is more than about 20 nm.

2. The nucleic acid molecule of claim 1, wherein the bCAR comprises an extracellular domain comprising a single-chain binding domain, a rigid elongation domain and a substrate for a sheddase, and further comprises an intracellular domain comprising a full or partial intracellular domain of a membrane phosphatase capable of dephosphorylating phosphorylated ITAMs.

3. The nucleic acid molecule of claim 1 or 2, wherein the sheddase is a disintegrin and metalloproteinase (ADAM) or a beta-secretase 1 (BACE1).

4. The nucleic acid molecule of claim 3, wherein the substrate comprises Lin 12/Notch repeats and an ADAM protease cleavage site.

5. The nucleic acid molecule of claim 1 or 2, wherein the membrane phosphatase capable of dephosphorylating phosphorylated ITAMs is CD45 or CD148.

6. The nucleic acid molecule of claim 1, wherein the bCAR comprises an extracellular domain comprising a single-chain binding domain and a rigid elongation domain, and further comprises an intracellular domain comprising a module capable of association or colocalization with TCR-CD3 complex.

7. The nucleic acid molecule of claim 6, wherein the module capable of association or colocalization with TCR is the intracellular domain of native CD2.

8. The nucleic acid molecule of claim 1, wherein the binding domain comprises (i) a single chain variable fragment (scFv); a functional fragment of an antibody; a single-domain antibody, such as a Nanobody; and a recombinant antibody; (ii) an antibody mimetic, such as an affibody molecule; an affilin; an affimer; an affitin; an alphabody; an anticalin; an avimer; a DARPin; a fynomer; a Kunitz domain peptide; and a monobody; (iii) an aptamer; or (iv) a binding domain of a native receptor, such as a cytokine receptor or a ligand such as a cytokine.

9. The nucleic acid molecule of claim 1, wherein the rigid elongation domain comprises at least one rigid protein module, such as a fibronectin type III repeat or an Ig domain harboring the typical motifs of the Ig fold.

10. The nucleic acid molecule of claim 9, wherein the rigid elongation domain essentially consists of the extracellular domain of CD45 (comprising three fibronectin type III repeats) or CD148 (comprising five fibronectin type III repeats) or a fragment thereof (comprising e.g. two or three fibronectin type III repeats); and is optionally linked to the extracellular or transmembrane domain of CD2, CD4, or CD8, or a fragment thereof.

11. The nucleic acid molecule of claim 10, wherein the elongation domain is linked to the extracellular and/or transmembrane domain of CD2.

12. The nucleic acid molecule of claim 1, wherein the abnormal tissue is a pre-malignant tissue or a malignant tumor.

13. The nucleic acid molecule of claim 12, wherein the cell surface epitope present on normal but not on abnormal mammalian tissue is a single allelic variant of a polymorphic cell surface epitope not expressed by the abnormal mammalian tissue but present on all cells of related mammalian normal tissue.

14. The nucleic acid molecule of claim 13, wherein the cell surface epitope is selected from the group consisting of cell surface epitopes of essential tissue-associated or housekeeping gene products, such as an ion channel or a receptor tyrosine kinase.

15. The nucleic acid molecule of claim 1, wherein the mammalian tissue being targeted by an autoimmune response is selected from the group consisting of pancreatic islets of the pancreas; digestive system; small intestine; large intestine; colon; thyroid; nervous system; skin, thyroid, and articular joints.

16. The nucleic acid molecule of claim 1, wherein the mammalian tissue is human tissue.

17. The nucleic acid molecule of claim 1, wherein the undesired activation of an immune cell is activation caused by specific binding of the immune cell to normal tissue via one or more cell-surface epitopes present on normal and related abnormal mammalian tissue or mammalian tissue being targeted by an autoimmune response, wherein the undesired activation is prevented by specific binding of the binding domain of the bCAR to another cell surface epitope present on normal but not on related abnormal mammalian tissue or mammalian tissue being targeted by an autoimmune response.

18. The nucleic acid molecule of claim 1, wherein the immune cell is an effector immune cell, such as an effector T cell; or a regulatory immune cell, such as a regulatory T cell.

19. The nucleic acid molecule of claim 1, wherein the binding domain is a scFv and the elongation domain comprises two or three fibronectin type III repeats, e.g. within the sequence of a full or partial extracellular domain of CD45 or CD148, optionally linked to the extracellular or transmembrane domain of CD2, CD4 or CD8, or a fragment thereof.

20. The nucleic acid molecule of claim 1, wherein the binding domain is a scFv and the substrate for a sheddase is selected from a disintegrin and metalloproteinase (ADAM) and a beta-secretase 1 (BACE1), e.g. a substrate comprising Lin 12/Notch repeats and an ADAM protease cleavage site.

21. The nucleic acid molecule of claim 1, wherein the binding domain is a scFv and the intracellular domain is the full or partial intracellular domain of CD45, CD148 or CD2.

22. The nucleic acid molecule of claim 1, wherein the binding domain is a scFv, the elongation domain comprises three fibronectin type III repeats, e.g. within the sequence of a full or partial extracellular domain of CD45 or CD148, and the substrate for a sheddase is selected from a disintegrin and metalloproteinase (ADAM) and a beta-secretase 1 (BACE1), e.g. a substrate comprising Lin 12/Notch repeats and an ADAM protease cleavage site.

23. The nucleic acid molecule of claim 1, wherein the binding domain is a scFv, the elongation domain comprises two or three fibronectin type III repeats, e.g. within the sequence of a full or partial extracellular domain of CD45 or CD148, optionally linked to the extracellular or transmembrane domain of CD2, or a fragment thereof, and the intracellular domain is the full or partial intracellular domain of CD45, CD148 or CD2.

24. The nucleic acid molecule of claim 1, wherein the binding domain is a scFv, the elongation domain comprises three fibronectin type III repeats, e.g. within the sequence of a full or partial extracellular domain of CD45 or CD148, the substrate for a sheddase is selected from a disintegrin and metalloproteinase (ADAM) and a beta-secretase 1 (BACE1), e.g. a substrate comprising Lin 12/Notch repeats and an ADAM protease cleavage site, the intracellular domain is the full or partial intracellular domain of CD45 or CD148, and the length of the extracellular domain is more than about 20 nm.

25. The nucleic acid molecule of claim 1, wherein the binding domain is a scFv, the elongation domain comprises two fibronectin type III repeats linked to the extracellular or transmembrane domain of CD2, or a fragment thereof, the intracellular domain is the full intracellular domain of CD2, and the length of the extracellular domain is more than about 20 nm.

26. A nucleic acid molecule comprising a nucleotide sequence encoding a short blocking chimeric antigen receptor (sbCAR) capable of preventing undesired activation of an immune cell, wherein the sbCAR comprises an extracellular domain comprising a single-chain binding domain and an elongation domain, and an intracellular domain comprising a full or partial intracellular domain of a membrane phosphatase capable of dephosphorylating phosphorylated ITAMs, wherein the undesired activation of the immune cell is activation caused by specific binding of the immune cell to normal tissue via one or more cell-surface epitopes present on normal and abnormal mammalian tissue or mammalian tissue being targeted by an autoimmune response, and the binding domain specifically binds to another cell surface epitope present on abnormal mammalian tissue or mammalian tissue being targeted by an autoimmune response but not on normal mammalian tissue, and the length of the extracellular domain is less than about 20 nm and the total length of the extracellular domain and the cell surface epitope is more than about 20 nm.

27. The nucleic acid molecule of claim 26, wherein the elongation domain comprises one fibronectin type III repeat, e.g. within the sequence of a partial extracellular domain of CD45 or CD148, or an Ig-domain, and/or the membrane phosphatase capable of dephosphorylating phosphorylated ITAMs is CD45 or CD148.

28. The nucleic acid molecule of claim 26 or 27, wherein the undesired activation of an immune cell is activation caused by specific binding of the immune cell to normal tissue via a first cell-surface epitopes present on normal and related abnormal mammalian tissue or related mammalian tissue being targeted by an autoimmune response, wherein the undesired activation is prevented by specific binding of the binding domain of the sbCAR to a second cell surface epitope which in combination with the first cell surface epitope is present on abnormal tissue or mammalian tissue being targeted by an autoimmune response but not on related normal tissue.

29. A vector comprising (i) a nucleic acid molecule of any one of claims 1 to 14 and 16 to 25; (ii) a nucleic acid molecule of any one of claims 26 to 28; or (iii) a nucleic acid molecule of any one of claims 1 to 11 and 15 to 25; and at least one control element, such as a promoter, operably linked to the nucleic acid molecule.

30. An effector immune cell, such as an effector T cell, that specifically binds to and is potentially activated by a first cell surface epitope shared by cells of related abnormal and normal mammalian tissue, wherein the effector immune cell comprises a nucleic acid molecule comprising a nucleotide sequence of any one of claims 1 to 14 and 16 to 25 encoding a bCAR that specifically binds to a second cell surface epitope present on normal mammalian tissue but not on related abnormal mammalian tissue, or a vector of claim 29(i); or a nucleic acid molecule comprising a nucleotide sequence of any one of claims 26 to 28 encoding an sbCAR that specifically binds to a second cell surface epitope which in combination with the first cell surface epitope is present on abnormal tissue but not on related normal tissue, or a vector of claim 29(ii).

31. The effector immune cell of claim 30, further comprising a nucleic acid molecule comprising a nucleotide sequence encoding an activating chimeric antigen receptor (aCAR) polypeptide providing the specific binding to, and potential activation by, the first cell surface epitope.

32. A regulatory immune cell, such as a regulatory T cell, that specifically binds to and is potentially activated by a cell surface epitope on a mammalian tissue being targeted by an autoimmune response, wherein the regulatory immune cell comprises a nucleic acid molecule comprising a nucleotide sequence of any one of claims 1 to 11 and 15 to 28 or a vector of claim 29(iii).

33. A method for treating cancer comprising administering to a cancer patient immune effector cells of claim 30 or 31.

34. The method of claim 32, wherein the immune effector cells are T cells, natural killer cells or cytokine-induced killer cells.

35. The method of claim 33 or 34, wherein the immune effector cells are universal (allogeneic) effector cells.

36. A method of treating an autoimmune disease comprising administering to an autoimmune disease patient regulatory immune cells of claim 32.

37. The method of claim 36, wherein the regulatory immune cells are universal (allogeneic) effector cells, such as universal regulatory T cells.

### EXAMPLES

### Example 1—Elongated bCAR Inhibition of aCAR-Induced T-Cell Activity in an Antigen-Dependent Manner

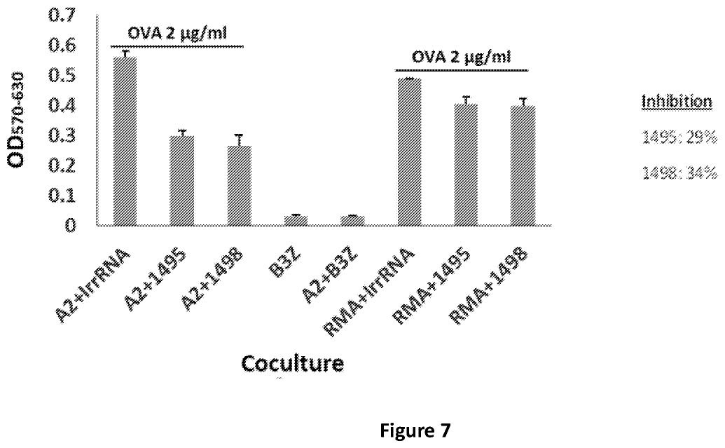

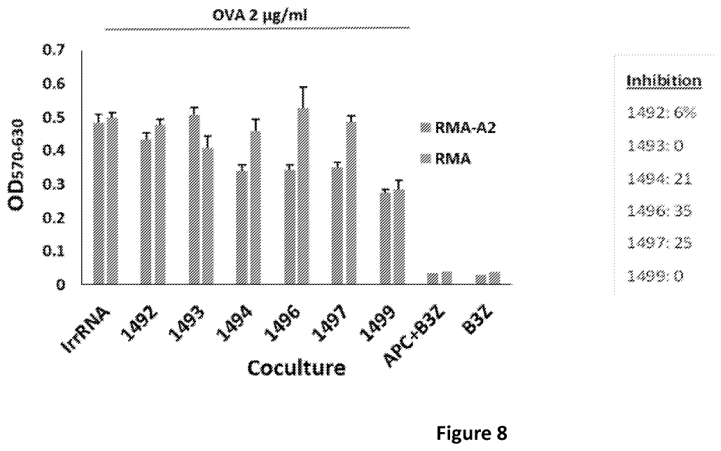

To explore the ability of “elongated” bCARs to prevent antigen-dependent aCAR-induced T-cell activity, several bCARs were designed, as depicted in FIGS. 6A and B. Such exemplary elongated bCARs, include an anti-HLA-A2 scFv, an extracellular domain that includes extracellular domain components of CD22 and CD2, or CD45 and a CD2 transmembrane and intracellular domain component. The reporter B3Z T cells were electroporated with in-vitro-transcribed mRNA encoding the indicated bCAR constructs or irrelevant mRNA as negative control and then cocultured with RMA or RMA-A2, loaded with 2 μg/ml OVA peptide. Following 8-12 hours coculture the level of B3Z activation was monitored via the colorimetric CPRG assay. As shown in FIGS. 7 and 8, several exemplary elongated bCARs were capable of inhibiting antigen-dependent aCAR-induced T cell activity in an antigen-dependent manner.

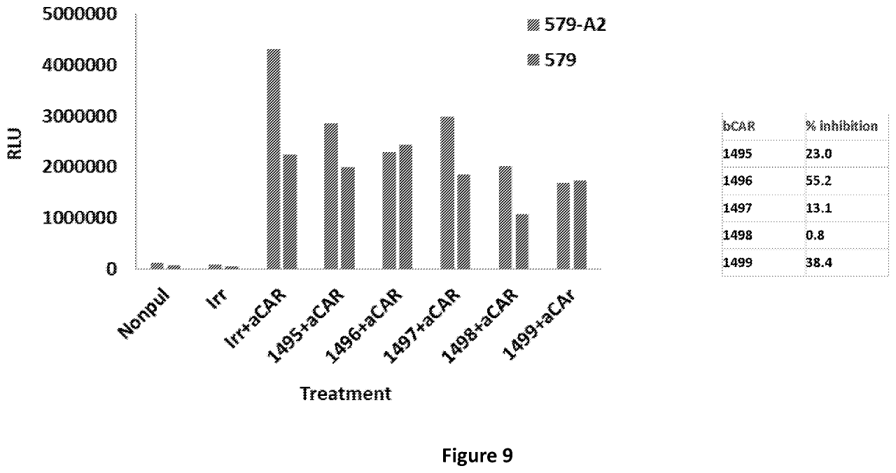

In a similar experiment, human Jurkat T cells harboring the NF-kB-luciferase reporter gene were electroporated with 10 mg of mRNA encoding an anti-melanotransferrin aCAR and 10 mg mRNA of each of the indicated anti-HLA-A2 bCARs (1495-1499) or irrelevant mRNA (Irr). Eight hours later transfected cells were mixed at 1:1 ratio with human melanoma cells 579 (HLA-A2(−) or 579-A2 (a stable transfectant of 579 cells expressing HLA-A2, both expressing melanotransferrin at identical levels) and incubated overnight. Following co-culture cell lysates were prepared and luciferase activity in each sample was monitored with a luminometer. The results in FIG. 9 shows again that the bCAR is capable of inhibiting T cell activity in an antigen-dependent manner.

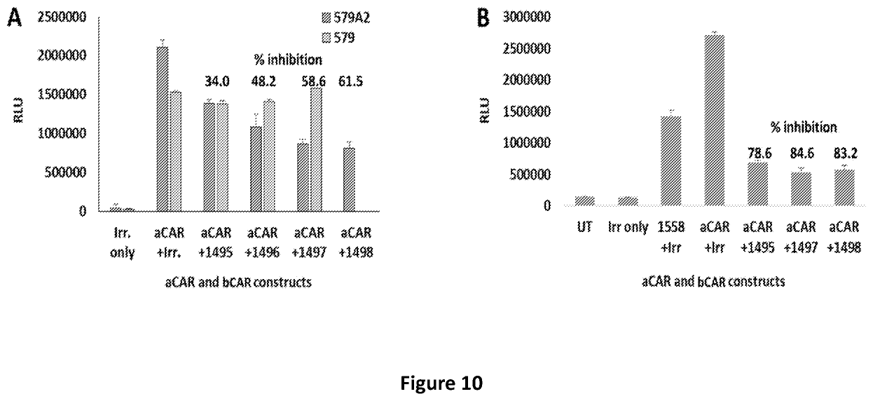

In another experiment, NFAT-Luc reporter Jurkat cells were co-electroporated with mRNA encoding the anti-MTf aCAR and the indicated anti-A2 bCARs or irrelevant mRNA (Irr.) at 1:4 mRNA ratio. Cells were then co-incubated with 579 or 579-A2 melanoma cells and cell lysates were then assayed for luciferase activity using a luminometer. FIGS. 10A and B show the results of two independent experiments. RLU, relative luminescence units. Percent inhibition was calculated relative to the RLU value recorded for Irr. mRNA, where Irr. mRNA only serves as background. 1558 is a conventional anti-A2 activating CAR harboring the same scFv and served control for T cell activation.

When comparing the effect of the different bCARs to that of irrelevant mRNA (FIG. 10A, orange histograms), no inhibition is evident in the absence of the HLA-A2 bCAR target. Of note, in the second experiment (FIG. 10B) the two longer bCARs, 1497 and 1498, showed >80% inhibition.

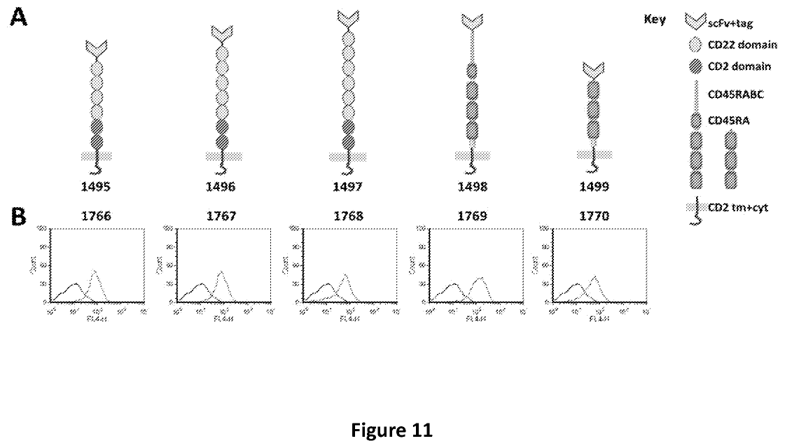

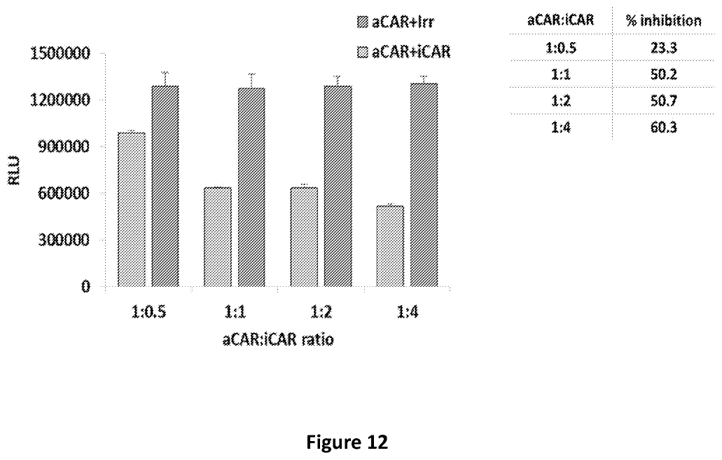

Anti-melanotransferrin (MTf) elongated bCARs where made and tested for inhibitory activity (FIGS. 11A and B). First, the inhibitory effect of bCAR 1766, which harbors six Ig domains, was assessed at different aCAR to bCAR ratios. For this purpose Jurkat NFκB-Luciferase reporter T cells were used as effectors and HLA-A2(+) MTf(+) 579-A2 melanoma cells were used as targets. The anti-HLA-A2 aCAR CD28-γ CAR 1558, which we was used. The amount of aCAR mRNA and the total amount of mRNA used for electroporation remained constant throughout the entire experiment (complementing the required mRNA quantity with irrelevant mRNA). FIG. 12 show that bCAR 1766 is capable of inhibiting T cell activity in an antigen-dependent manner.

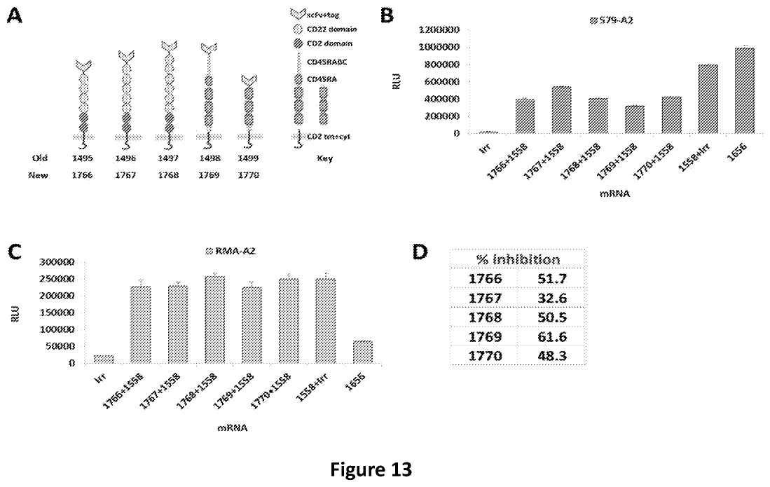

Additional anti-melanotransferrin (MTf) elongated bCARs where made and tested for inhibitory activity (FIG. 13). In this experiment, Jurkat cells were co-transfected with an mRNA encoding the anti-HLA-A2 aCAR 1558 along with mRNA coding for the series of the anti-MTf bCARs (depicted in FIG. 13A) or with Irr. RNA. These Jurkat cell transfectants were then cocultured with 579-A2 melanoma cells, which express both HLA-A2 and MTf (FIG. 13B). To confirm that inhibition is antigen-specific, these cells were co-cultured in parallel with RMA-A2 cells, which express HLA-A2 but do not express human MTf, so that no inhibition was expected (FIG. 13C). The degree of inhibition achieved by each of the bCARs tested is shown in FIG. 13D. As shown in FIG. 13D, the additional anti-melanotransferrin (MTf) elongated bCARs are capable of inhibiting T cell activity in an antigen-dependent manner.

### Example 2—Elongated bCAR with Variant Intracellular CD2 Domains

CD2 possesses an inherent co-stimulatory activity manifested in the induction of calcium influx and downstream signaling, which is ligand-dependent. Moingeon et al., Immuno. Rev. 111:111-144 (1989); and Cheadle et al., Gene Ther. 19:1114-1120 (2012). To further increase the inhibitory capacity of the subject bCARs, bCARs with CD2 variant intracellular domains were made to uncouple CD2-mediated costimulatory signaling from binding of the CD2 moiety to the aTCR/aCAR through the actin cytoskeleton. The ability to activate CD2-mediated costimulatory signaling is attributed to two identical proline-rich stretches: PPPGHR within the CD2 intracellular domain. Mutation/deletion of these proline-rich stretches completely abolish this signaling. Moreover, these stretches were found to bind an intracellular protein termed CD2 binding protein 2 (CD2BP2). Nishizawa et al., Proc. Natl. Acad. Sci. 95: 14897-14902 (1998). Further, association of CD2 with the TCR/CD3 complex via the actin cytoskeleton is accomplished by the CD2-adaptor protein CD2AP (also known as CMS. Dustin et al., Cell 94:667-677 (1998); and Lehtonen, Am. J. Physiol. 283:F734-F743 (2002). The original work that identified CD2AP (Dustin et al., Cell 94:667-677 (1998)) has demonstrated that binding of the CD2AP SH3-1 domain to murine CD2 takes place via the C-terminal proline-rich region in the CD2 intracellular domain. In particular, mutagenesis data demonstrated that the primary binding site is a Type II SH3 ligand (PPLPRPR) and five to seven C-terminal flanking residues to the motif are required for binding. But, see Kurakin et al., J. Biol. Chem. 278:34102-9 (2003) for additional analysis concerning this binding site.

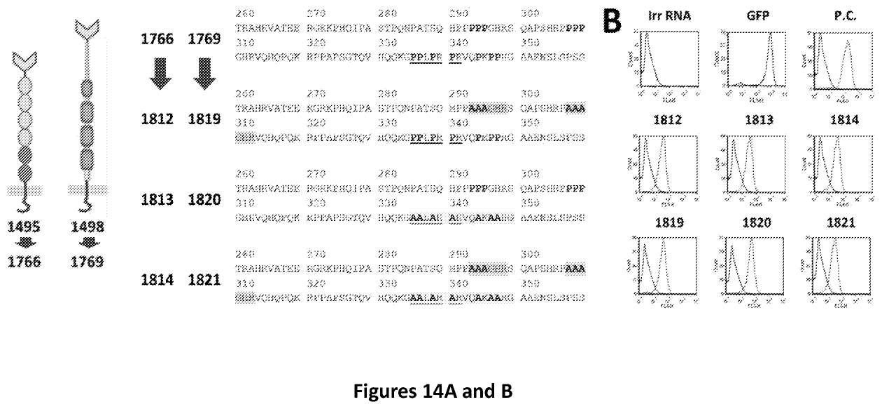

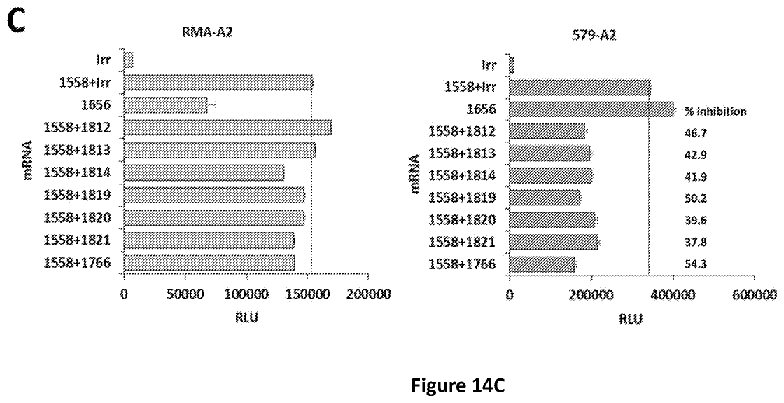

A schematic representation of these mutants and their designations are shown in FIG. 14A. All these constructs harbor the anti-MTf scFv and the Ha tag and are based on either the original 1495 or 1498 iCAR constructs and mutations and designations are specified. High surface expression of all these constructs following mRNA transfection of K562 cells is shown in FIG. 14B. The ability of the different mutants to inhibit the activation of NFAT-Luciferase reporter Jurkat cells co-transfected with an a CAR specific to HLA-A2 (1558) upon coculture with RMA-A2 cells (MTf(−)) or 579-A2 melanoma cells (MTf(+) is shown in FIG. 14C. While no clear inhibition was evident for the iCARs in the absence of the inhibitory antigen (the MTf(−) RMA-A2 cells) inhibition ranging between ˜40% and 55% was evident for all of the iCARs tested. No reproducible effect could be ascribed to the two mutations or their combinations as examined here. Of note, an unexpected response of Jurkat cells expressing the 1656 aCAR against the MTf(−) RMA-A2 cells could be observed.

### Example 3—bCARs Based on SynNotch

According to the KS model, and as has been shown in a previous study which had investigated shortened versions of CD45, preventing the exclusion of the CD45 phosphatase from the contact zone exerts a strong inhibitory effect on T cell activation. We hypothesize that the expression of an elongated CAR-like receptor harboring an extracellular antigen-binding domain and the intracellular CD45 phosphatase moiety will similarly inhibit T cell activation if the extracellular domain is cleaved upon antigen binding. Similarly to the CD2-based bCARs, such a mode of action would couple two inhibitory effect: retention of the phosphatase activity at the contact zone and prevention of aCAR (or aTCR) binding to its antigen.

### Goal

Develop a conditionally cleavable bCAR that will retain membrane attachment.

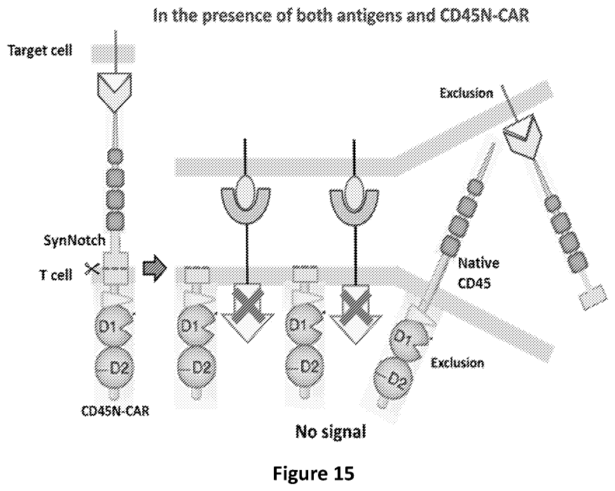

The genetic platform we are exploring is based on the documented ability of a minimal synthetic Notch receptor (SynNotch) to induce regulated intramembrane proteolysis (RIP) of artificially fused extra- and intracellular domains. In this platform an antigen-binding ectodomain and the CD45 phosphatase intracellular domain are genetically tethered to SynNotch so that that antigen binding will only induce cleavage of the extracellular, but not the intracellular portion, thereby assuring that the phosphatase activity is retained attached to the cell membrane at the contact zone (FIG. 15).

For preventing cleavage of the intracellular portion we are exploiting two known mutations, one in transmembrane domain of human Notch1 and the other in the membrane-proximal region of the Notch1 endodomain. The meticulous analysis of amino acid alterations which affect γ-secretase cleavage of amyloid precursor protein in Alzheimer Disease and additional substrates, including human Notch1 (Cell Discov. 2016), has provided us with precise information on the actual mutations which can confer the desired resistance to cleavage. In particular, the effect of two mutations are explored, separately and combined: L1746Y and R1758E (see below, underlined purple).

| | Ecto- | | Intra- |
|---|---|---|---|
| | domain | Transmembrane | cellular |
| Mouse Notch1 | PSQLH | LMYVAAAAFVLLFFVGCGV | SRKRRR |
| (UniProtKB | | LL | |
| Q01705): | | | |
| Human Notch1 | PAQLH | FMYVAAAAFVLLFFVGCGV | SRKRRR |
| (UniProtKB | | LL | |
| P46531): | | | |
| Mutated Human | PAQLH | FMYVAAAAFVYLFFVGCGV | SEKRRR |
| Notch 1 (35): | | LL | |

### Results

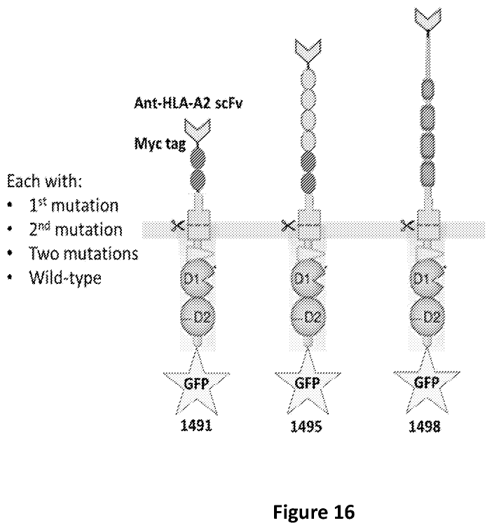

Following the genetic scheme delineated above, four SynNotch-iCAR gene segments have been assembled, all possessing the same minimal Notch-1 extracellular sequence but different transmembrane and intracellular sequences: wild type (no mutations), a single L1746L or R1758E mutation or both L1746Y+R1758E mutations. Each of these segments served as backbone for the construction of three bCARs, all harboring the same anti-HLA-A2 scFv followed by the Myc tag and the intracellular CD45 phosphatase portion but different extracellular spacer, derived from the 1491, 1495 or 1498 constructs (see FIG. 6A). Full length EGFP was fused to the C-terminus of all these constructs to allow detection of the intracellular domains. All these constructs were cloned in the mammalian expression vector pBJ1-Neo to allow transient or stable expression in selected cell lines. Scheme of these constructs are depicted in FIG. 16.

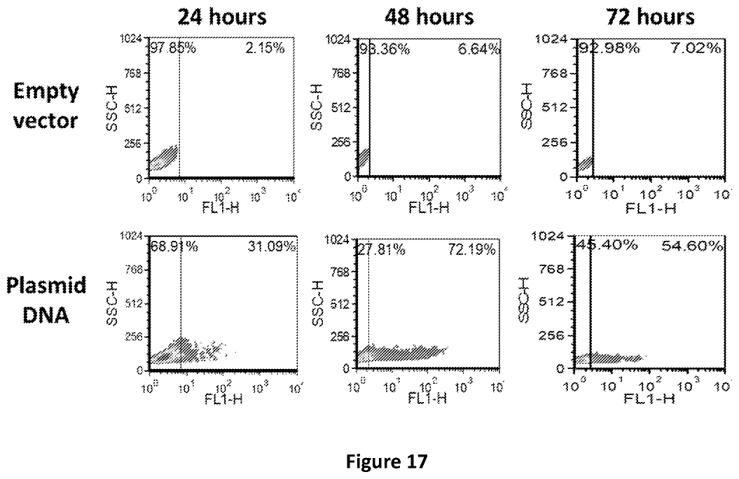

As a preparatory experiment for assessing proper expression of these constructs we assessed GFP fluorescence of one of these constructs in transiently transfected HEK293T cells (FIG. 17).

### Objective

Obtain POC for the proposed inhibitory activity of SynNotch-based bCARs. Specifically this example is directed to confirming the anticipated single-cleavage of our mutated SynNotch constructs, determining the inhibitory capacity of SynNotch-based bCARs, and examining the experimental system and research plan

We will perform a series of experiments in transiently transfected HEK293T cells, as, unlike T cells, these are easily transfectable with plasmid DNA and express the enzymatic machinery required for the proposed RIP. We will first employ co-localization assay using confocal microscopy to confirm membrane expression of the different constructs. To this end we will stain transiently transfected HEK293T cells 48 hours post-transfection (see FIG. 17) with anti-Myc tag Abs and assess merging with EGFP fluorescence. We will then perform co-culture experiments in which HEK293T transfectants will be incubated with pre-stained 579 or 579-A2 melanoma cells. It is expected that incubation with 579-A2, but not with 579 cells, will result in single cleavage which will liberate the entire extracellular domain of the respective bCAR that will remain bound to its HLA-A2 target antigen at the membrane of the melanoma cells. This outcome can be assessed by both flow cytometry and confocal microscopy. Using the same confocal microscopy analysis, this experimental setting will also allow us to determine whether engagement results in single cleavage (retaining EGFP at the cell surface) or double cleavage (releasing EGFP to the cytosol).

We will generate stable transfectants of the NFAT-Luc reporter Jurkat cells (employing puromycin selection) with a limited number of constructs selected according to the results obtained with transiently transfected HEK293T cells. Selection of puromycin-resistant clones for construct expression will exploit EGFP fluorescence. Having these stable Jurkat transfectants made, we will use mRNA transfection of the anti-MTf aCAR and functional assays following co-culture with the 579 and 579-A2 melanoma cells will be performed essentially as described above for the CD2-based bCARs.

### Discussion

SynNotch bCARs

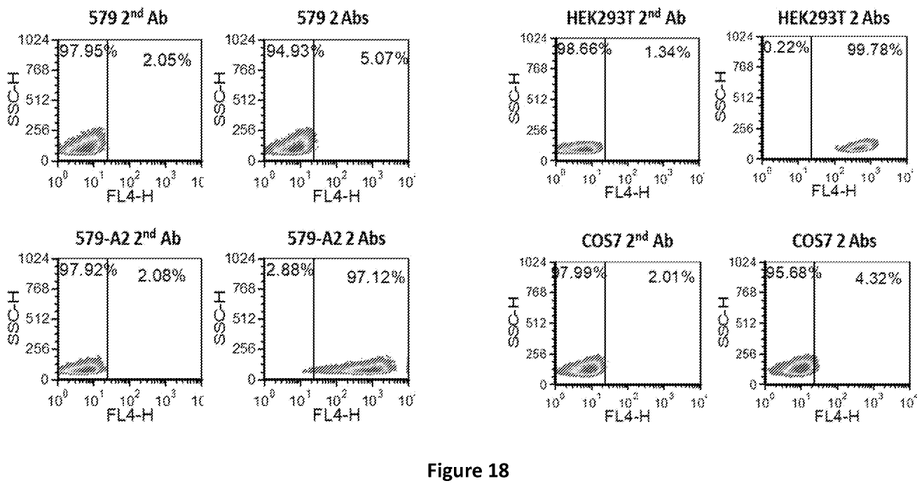

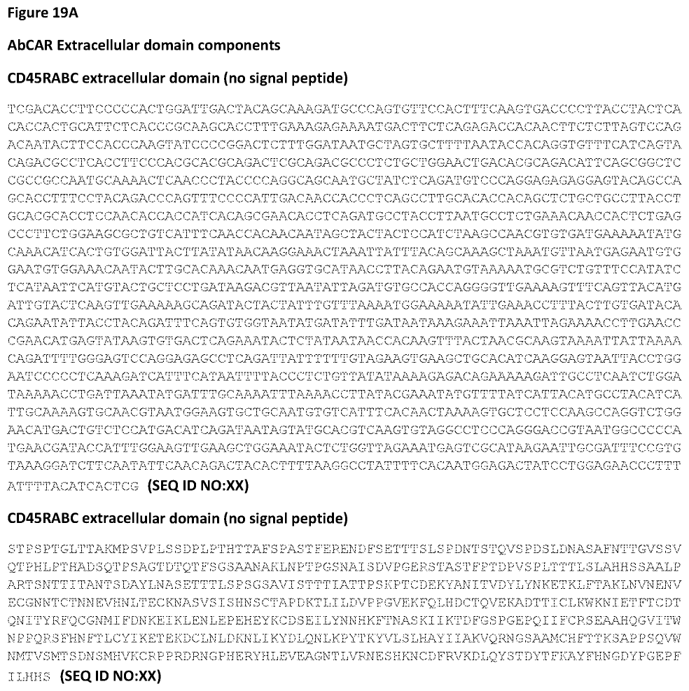

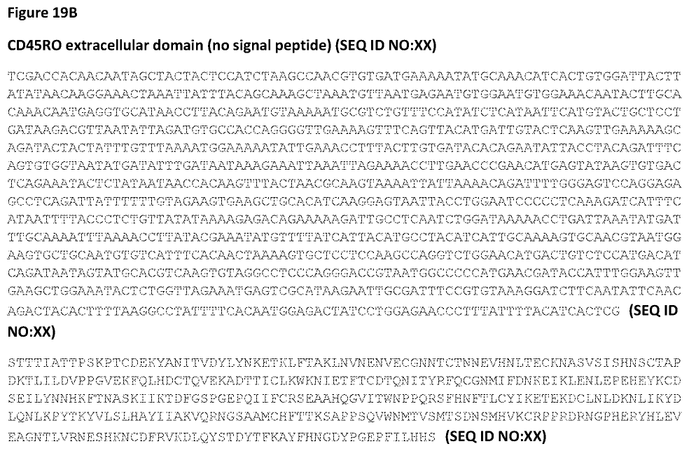

The HEK293T system was chosen for preliminary analysis of expression and anticipated single cleavage of these receptors. As all our SynNotch bCARs are directed at HLA-A2, employing an HLA-A2-negative cell line was required for these experiments. Looking for a candidate we were guided by a paper (Dellgren, C., J. O. et al., 2015. PLoS One 10: e0135385) claiming that: “The HEK293T HLA type was confirmed as being homozygous for the haplotype: HLA-A*03; HLA-B*07; HLA-C*07 . . . ”, and consequently, negative for HLA-A2. However, we later came across another paper (Vogel, R., R. et al., 2013. Mass Spectrometry Reveals Changes in MHC I Antigen Presentation After Lentivector Expression of a Gene Regulation System. Mol. Ther.-Nucleic Acids 2: e75) arguing that “HEK 293T cells were HLA-A*02/02, HLA-B*07/07, HLA-C*07/07 . . . ”. We analyzed this further and decided to determine the HLA-A2 status of these cells directly, using the same BB7.2 mAb, which served for the construction of all of our anti-HLA-A2 CARs. In parallel it was verified that the BB7.2 mAb does not cross-react with any of the MHC-I molecules expressed by the COS7 monkey cell line, another easily transfectable candidate expressing the SV40 large T antigen. The results are shown in FIG. 18 and clearly confirm that HEK293T cells are strongly stained by BB7.2. No cross-reactivity was detected with COS7 cells and we will continue assessing the series of SynNotch iCARs with these cells.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A polynucleotide encoding a blocking chimeric; antigen receptor (bCAR) capable of preventing undesired activation of an immune cell comprising:
a. an extracellular domain that is more than about 20 nm in length comprising:
i. a single-chain binding domain,
ii. a rigid elongation domain, and
iii. a protease cleavage site capable of being cleaved by a protease;
b. a transmembrane domain; and
c. intracellular domain comprising a full or partial intracellular domain of a membrane phosphatase capable of dephosphorylating phosphorylated immunoreceptor tyrosine-based activation motifs (ITAMs),
wherein the protease cleavage site is linked to the intracellular domain by the transmembrane domain,
wherein the binding domain specifically binds to a cell surface epitope present on normal mammalian tissue, but absent on related abnormal mammalian tissue or mammalian tissue being targeted by an autoimmune response.

2. The polynucleotide according to claim 1, wherein the protease is a disintegrin metalloproteinase (ADAM) or a beta-secretase 1 (BACE1).

3. The polynucleotide according to claim 2, wherein the protease cleavage site comprises Lin 12/Notch repeats and/or an ADAM protease cleavage site.

4. The polynucleotide according to claim 1, wherein the protease is a disintegrin and metalloproteinase (ADAM) or a beta-secretase 1 (BACE1).

5. (canceled)

6. (canceled)

7. The polynucleotide according to claim 1, wherein the binding domain comprises (i) a single chain variable fragment (scFv); a functional fragment of an antibody; a single domain antibody, such as a Nanobody; and a recombinant antibody.

8. The polynucleotide according to claim 1, wherein the membrane phosphatase capable of dephosphorylating phosphorylated ITAMs is CD45 or CD148.

9. The polynucleotide according to claim 1, wherein the rigid elongation domain comprises at least one rigid protein module.

10. The polynucleotide according to claim 9, wherein the at least one rigid protein module comprises a fibronectin type III repeat or an Ig domain comprising an Ig fold motif.

11. The polynucleotide according to claim 9, wherein the at least one rigid protein module comprises the extracellular domain of CD45 or CD148 or a fragment thereof.

12. The polynucleotide according to claim 9, wherein the rigid elongation domain is linked to the extracellular or transmembrane domain of CD2, CD4, or CD8, or a fragment thereof.

13. The polynucleotide according to claim 11, wherein the elongation domain is linked to the extracellular and/or transmembrane domain of CD2.

14. The polynucleotide according to claim 1, wherein the abnormal tissue is a pre-malignant tissue or a malignant tumor.

15. The polynucleotide according to claim 13, wherein the cell surface epitope is a single allelic variant of a polymorphic cell surface epitope not expressed by the abnormal mammalian tissue but present on all cells of related mammalian normal tissue.

16. The polynucleotide according to claim 15, wherein the cell surface epitope is a cell surface epitope of an essential tissue-associated gene product or housekeeping gene products.

17. The polynucleotide according to claim 16, wherein the cell surface epitope is an ion channel or a receptor tyrosine kinase.

18. The polynucleotide according to claim 17, wherein the mammalian tissue being targeted by an autoimmune response is selected from the group consisting of pancreatic islets of the pancreas; digestive system; small intestine; large intestine; colon; thyroid; nervous system; skin, thyroid, and articular joints.

19. The polynucleotide according to claim 18, wherein the mammalian tissue is human tissue.

20. The polynucleotide according to claim 19, wherein the undesired activation of an immune cell is activation caused by specific binding of the immune cell to normal tissue via one or more cell-surface epitopes present on normal and related abnormal mammalian tissue or mammalian tissue being targeted by an autoimmune response.

21. The polynucleotide according to claim 20, wherein the immune cell is an effector immune cell, such as an effector T cell; or a regulatory immune cell, such as a regulatory T cell.

22. The polynucleotide according to claim 1,
wherein the binding domain is an scFv
wherein the elongation domain comprises an extracellular domain of a CD45 or a CD148 or fragment thereof,
wherein the elongation domain comprises a plurality of fibronectin repeats, and wherein the elongation domain is optionally linked to an extracellular domain or transmembrane domain or fragment thereof of CD2, CD4 or CD8.

23. The polynucleotide according to claim 1,
wherein the binding domain is an scFv, and
wherein the protease is a disintegrin and metalloproteinase (ADAM) or a beta-secretase 1 (BACE1).

24. The polynucleotide according to claim 1, wherein the binding domain is a scFv and the intracellular domain is an intracellular domain of CD45, CD148 or CD2 or fragment thereof.

25. The polynucleotide according to claim 1,
wherein the binding domain is an scFv,
wherein the elongation domain comprises an extracellular domain of a CD45 or a CD148 or fragment thereof,
wherein the elongation domain comprises a plurality of fibronectin repeats, and wherein the protease is a disintegrin and metalloproteinase (ADAM) or a beta-secretase 1 (BACE1).

26. The polynucleotide according to claim 1,
wherein the binding domain is an scFv,
wherein the elongation domain comprises an extracellular domain of a CD45 or a CD148 or fragment thereof,
wherein the elongation domain comprises a plurality of fibronectin repeats, wherein the elongation domain is optionally linked to a CD2 extracellular domain or transmembrane domain or fragment thereof, and
wherein the intracellular domain is a CD45, CD148 or CD2 intracellular domain or fragment.

27. The polynucleotide according to claim 1,
wherein the binding domain is an scFv,
wherein the elongation domain comprises an extracellular domain of a CD45 or a CD148 or fragment thereof,
wherein the elongation domain comprises a plurality of fibronectin repeats, wherein the protease is a disintegrin and metalloproteinase (ADAM) or a beta-secretase 1 (BACE1), and
wherein the intracellular domain is a CD45 or CD148 intracellular domain or fragment thereof.

28. (canceled)

29. A polynucleotide encoding a short blocking chimeric antigen receptor (sbCAR) capable of preventing undesired activation of an immune cell comprising:
a. an extracellular domain that is less than about 20 nm in length comprising:
i. a single-chain binding domain, and
ii. an elongation domain;
b. a transmembrane domain; and
c. an intracellular domain comprising a full or partial intracellular domain of a membrane phosphatase capable of dephosphorylating phosphorylated immunoreceptor tyrosine-based activation motifs (ITAMs),
wherein the undesired activation of the immune cell is activation caused by specific binding of the immune cell to normal tissue via one or more cell-surface epitopes present on normal and abnormal mammalian tissue or mammalian tissue being targeted by an autoimmune response,
wherein the binding domain specifically binds to another cell surface epitope present on abnormal mammalian tissue or mammalian tissue being targeted by an autoimmune response but not on normal mammalian tissue
wherein the undesired activation of the immune cell is activation caused by specific binding of the immune cell to normal tissue via one or more cell-surface epitopes present on normal and abnormal mammalian tissue or mammalian tissue being targeted by an autoimmune response,
wherein the total length of the extracellular domain and the cell surface epitope is more than about 20 nm.

30. The polynucleotide according to claim 29, wherein the elongation domain comprises a fibronectin type III repeat.

31. The polynucleotide according to claim 29, wherein the elongation domain comprises a partial extracellular domain of CD45 or CD 148 or an Ig-domain.

32. The polynucleotide according to claim 31, wherein the ITAM is CD45 or CD148.

33. The polynucleotide according to claim 32, wherein the undesired activation of an immune cell is activation caused by specific binding of the immune cell to normal tissue via a first cell-surface epitopes present on normal and related abnormal mammalian tissue or related mammalian tissue being targeted by an autoimmune response, wherein the undesired activation is prevented by specific binding of the binding domain of the sbCAR to a second cell surface epitope which in combination with the first cell surface epitope is present on abnormal tissue or mammalian tissue being targeted by an autoimmune response but not on related normal tissue.

34. An expression vector comprising the polynucleotide according to any one of claim 1 or 33; and at least one control element operably linked to the polynucleotide.

35. An effector immune cell, such as an effector T cell, that specifically binds to and is potentially activated by a first cell surface epitope shared by cells of related abnormal and normal mammalian tissue, wherein the effector immune cell comprises:
a. a polynucleotide according to any one of claim 1, 17 or 27, wherein the bCAR specifically binds to a second cell surface epitope present on normal mammalian tissue but not on related abnormal mammalian tissue; or
b. an expression vector comprising the polynucleotide according to any one of claims 1, 17 or 27.

36. An effector immune cell, such as an effector T cell, that specifically binds to and is potentially activated by a first cell surface epitope shared by cells of related abnormal and normal mammalian tissue, wherein the effector immune cell comprises:
a. a polynucleotide according claim 33, wherein the sbCAR specifically binds to a second cell surface epitope that in combination with the first cell surface epitope is present on abnormal tissue but not on related normal tissue, or
b. an expression vector comprising the polynucleotide according to claim 34.

37. The effector immune cell according to claim 36, further comprising a polynucleotide encoding an activating chimeric antigen receptor (aCAR) polypeptide providing the specific binding to, and potential activation by, the first cell surface epitope.

38. A regulatory immune cell, such as a regulatory T cell, that specifically binds to and is potentially activated by a cell surface epitope on a mammalian tissue being targeted by an autoimmune response, wherein the regulatory immune cell comprises:
a. a polynucleotide according to claim 1, 18, 19 or 29, wherein the bCAR specifically binds to a second cell surface epitope present on normal mammalian tissue but not on related abnormal mammalian tissue; or
b. an expression vector comprising the polynucleotide according to any one of claim 1, 18, 19 or 29.

39. (canceled)

40. (canceled)

41. (canceled)

42. (canceled)

43. (canceled)

44. A blocking chimeric antigen receptor (bCAR) capable of preventing undesired activation of an immune cell comprising:
a. an extracellular domain that is more than about 20 nm in length comprising:
i. a single-chain binding domain,
ii. a rigid elongation domain, and
iii. a protease cleavage site capable of being cleaved by a protease;
b. a transmembrane domain; and
c. an intracellular domain comprising a full or partial intracellular domain of a membrane phosphatase capable of dephosphorylating phosphorylated immunoreceptor tyrosine-based activation motifs (ITAMs),
wherein the protease cleavage site is linked to the intracellular domain by the transmembrane domain,
wherein the binding domain specifically binds to a cell surface epitope present on normal mammalian tissue, but absent on related abnormal mammalian tissue or mammalian tissue being targeted by an autoimmune response.

45. The bCAR according to claim 44, wherein the protease is a disintegrin and metalloproteinase (ADAM) or a beta-secretase 1 (BACE1).

46. The bCAR according to claim 44, wherein the protease cleavage site comprises Lin 12/Notch repeats and/or an ADAM protease cleavage site.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. The bCAR of claim 45, wherein the cell surface epitope is a single allelic variant of a polymorphic cell surface epitope not expressed by the abnormal mammalian tissue but present on all cells of related mammalian normal tissue.

60. The bCAR of claim 45, wherein the cell surface epitope is a cell surface epitope of an essential tissue-associated gene product or housekeeping gene products.

61. The bCAR of claim 60, wherein the cell surface epitope is an ion channel or a receptor tyrosine kinase.

62. The bCAR according to claim 45, wherein the mammalian tissue being targeted by an autoimmune response is selected from the group consisting of pancreatic islets of the pancreas; digestive system; small intestine; large intestine; colon; thyroid; nervous system; skin, thyroid, and articular joints.

63. The bCAR according to claim 62, wherein the mammalian tissue is human tissue.

64. The bCAR according to claim 63, wherein the undesired activation of an immune cell is activation caused by specific binding of the immune cell to normal tissue via one or more cell-surface epitopes present on normal and related abnormal mammalian tissue or mammalian tissue being targeted by an autoimmune response.

65. The bCAR according to claim 64, wherein the immune cell is an effector immune cell, such as an effector T cell; or a regulatory immune cell, such as a regulatory T cell.

66. The bCAR according to claim 65,
wherein the binding domain is an scFv
wherein the elongation domain comprises an extracellular domain of a CD45 or a CD148 or fragment thereof,
wherein the elongation domain comprises a plurality of fibronectin repeats, and wherein the elongation domain is optionally linked to an extracellular domain or transmembrane domain or fragment thereof of CD2, CD4 or CD8.

67. (canceled)

68. The bCAR according to claim 66, wherein the intracellular domain is an intracellular domain of CD45, CD148 or CD2 or fragment thereof.

69. The bCAR according to claim 44,
wherein the binding domain is an scFv,
wherein the elongation domain comprises an extracellular domain of a CD45 or a CD148 or fragment thereof,
wherein the elongation domain comprises a plurality of fibronectin repeats, and
wherein the protease is a disintegrin and metalloproteinase (ADAM) or a beta-secretase 1 (BACE1).

70. The bCAR according to claim 44,
wherein the binding domain is an scFv,
wherein the elongation domain comprises an extracellular domain of a CD45 or a CD148 or fragment thereof,
wherein the elongation domain comprises a plurality of fibronectin repeats, wherein the elongation domain is optionally linked to a CD2 extracellular domain or transmembrane domain or fragment thereof, and
wherein the intracellular domain is a CD45, CD148 or CD2 intracellular domain or fragment.

71. The bCAR according to claim 44,
wherein the binding domain is an scFv,
wherein the elongation domain comprises an extracellular domain of a CD45 or a CD148 or fragment thereof,
wherein the elongation domain comprises a plurality of fibronectin repeats, wherein the protease is a disintegrin and metalloproteinase (ADAM) or a beta-secretase 1 (BACE1), and
wherein the intracellular domain is a CD45 or CD148 intracellular domain or fragment thereof.

72. The bCAR according to claim 44,
wherein the binding domain is an scFv,
wherein the elongation domain comprises a plurality of fibronectin repeats, wherein the elongation domain is linked to a CD2 extracellular domain or transmembrane domain or fragment thereof, and
wherein the intracellular domain is a CD2 intracellular domain.

73. (canceled)

74. (canceled)

75. (canceled)

76. (canceled)

77. (canceled)


SEQUENCE LISTING (51 sequences)

SEQ ID NO: 1 - DNA, 1632 bp, Artificial Sequence
  CD45RABC extracellular domain
tcgacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcg

SEQ ID NO: 2 - protein, 544 aa, Artificial Sequence
  CD45RABC extracellular domain
STPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHS

SEQ ID NO: 3 - DNA, 1071 bp, Artificial Sequence
  CD45RO extracellular domain
tcgaccacaacaatagctactactccatctaagccaacgtgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacgttaatattagatgtgccaccaggggttgaaaagtttcagttacatgattgtactcaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgattaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcacgtcaagtgtaggcctcccagggaccgtaatggcccccatgaacgataccatttggaagttgaagctggaaatactctggttagaaatgagtcgcataagaattgcgatttccgtgtaaaggatcttcaatattcaacagactacacttttaaggcctattttcacaatggagactatcctggagaaccctttattttacatcactcg

SEQ ID NO: 4 - protein, 357 aa, Artificial Sequence
  CD45RO extracellular domain
STTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHS

SEQ ID NO: 5 - DNA, 2820 bp, Artificial Sequence
  CD148 Extracellular domain
gcaggtggcacccctagtccaattcctgacccttcagtagcaactgttgccacaggggaaaatggcataacgcagatcagcagtacagcagaatcctttcataaacagaatggaactggaacacctcaggtggaaacaaacaccagtgaggatggtgaaagctctggagccaacgatagtttaagaacacctgaacaaggatctaatgggactgatggggcatctcaaaaaactcccagtagcactgggcccagtcctgtgtttgacattaaagctgtttccatcagtccaaccaatgtgatcttaacttggaaaagtaatgacacagctgcttctgagtacaagtatgtagtaaagcataagatggaaaatgagaagacaattactgttgtgcatcaaccatggtgtaacatcacaggcttacgtccagcgacttcatatgtattctccatcactccaggaataggcaatgagacttggggagatcccagagtcataaaagtcatcacagagccgatcccagtttctgatctccgtgttgccctcacgggtgtgaggaaggctgctctctcctggagcaatggcaatggcactgcctcctgccgggttcttcttgaaagcattggaagccatgaggagttgactcaagactcaagacttcaggtcaatatctcgggcctgaagccaggggttcaatacaacatcaacccgtatcttctacaatcaaataagacaaagggagaccccttgggcacagaaggtggcttggatgccagcaatacagagagaagccgggcagggagccccaccgcccctgtgcatgatgagtccctcgtgggacctgtggacccatcctccggccagcagtcccgagacacggaagtcctgcttgtcgggttagagcctggcacccgatacaatgccaccgtttattcccaagcagcgaatggcacagaaggacagccccaggccatagagttcaggacaaatgctattcaggtttttgacgtcaccgctgtgaacatcagtgccacaagcctgaccctgatctggaaagtcagcgataacgagtcgtcatctaactatacctacaagatacatgtggcgggggagacagattcttccaatctcaacgtcagtgagcctcgcgctgtcatccccggactccgctccagcaccttctacaacatcacagtgtgtcctgtcctaggtgacatcgagggcacgccgggcttcctccaagtgcacaccccccctgttccagtttctgacttccgagtgacagtggtcagcacgacggagatcggcttagcatggagcagccatgatgcagaatcatttcagatgcatatcacacaggagggagctggcaattctcgggtagaaataaccaccaaccaaagtattatcattggtggcttgttccctggaaccaagtattgctttgaaatagttccaaaaggaccaaatgggactgaaggggcatctcggacagtttgcaatagaactgttcccagtgcagtgtttgacatccacgtggtctacgtcaccaccacggagatgtggctggactggaagagccctgacggtgcttccgagtatgtctaccatttagtcatagagtccaagcatggctctaaccacacaagcacgtatgacaaagcgattactctccagggcctgattccgggcaccttatataacatcaccatctctccagaagtggaccacgtctggggggaccccaactccactgcacagtacacacggcccagcaatgtgtccaacattgatgtaagtaccaacaccacagcagcaactttaagttggcagaactttgatgacgcctctcccacgtactcctactgccttcttattgagaaggctggaaattccagcaacgcaacacaagtagtcacggacattggaattactgacgctacagtcactgaattaatacctggctcatcatacacagtggagatctttgcacaagtaggggatgggatcaagtcactggaacctggccggaagtcattctgtacagatcctgcgtccatggcctccttcgactgcgaagtggtccccaaagagccagccctggttctcaaatggacctgccctcctggcgccaatgcaggctttgagctggaggtcagcagtggagcctggaacaatgcgacccacctggagagctgctcctctgagaatggcactgagtatagaacggaagtcacgtatttgaatttttctacctcgtacaacatcagcatcaccactgtgtcctgtggaaagatggcagcccccacccggaacacctgcactactggcatcacagatccccctcctccagatggatcccctaatattacatctgtcagtcacaattcagtaaaggtcaagttcagtggatttgaagccagccacggacccatcaaagcctatgctgtcattctcaccaccggggaagctggtcacccttctgcagatgtcctgaaatacacgtatgaggatttcaaaaagggagcctcagatacttatgtgacatacctcataagaacagaagaaaagggacgttctcagagcttgtctgaagttttgaaatatgaaattgacgttgggaatgagtcaaccacacttggttattacaatgggaagctggaacctctgggctcctaccgggcttgtgtggctggcttcaccaacattaccttccaccctcaaaacaaggggctcattgatggggctgagagctatgtgtccttcagtcgctactcagatgctgtttccttgccccaggatccaggtgtcatctgtgga

SEQ ID NO: 6 - protein, 940 aa, Artificial Sequence
  CD148 Extracellular domain
AGGTPSPIPDPSVATVATGENGITQISSTAESFHKQNGTGTPQVETNTSEDGESSGANDSLRTPEQGSNGTDGASQKTPSSTGPSPVFDIKAVSISPTNVILTWKSNDTAASEYKYVVKHKMENEKTITVVHQPWCNITGLRPATSYVFSITPGIGNETWGDPRVIKVITEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTVPSAVFDIHVVYVTTTEMWLDWKSPDGASEYVYHLVIESKHGSNHTSTYDKAITLQGLIPGTLYNITISPEVDHVWGDPNSTAQYTRPSNVSNIDVSTNTTAATLSWQNFDDASPTYSYCLLIEKAGNSSNATQVVTDIGITDATVTELIPGSSYTVEIFAQVGDGIKSLEPGRKSFCTDPASMASFDCEVVPKEPALVLKWTCPPGANAGFELEVSSGAWNNATHLESCSSENGTEYRTEVTYLNFSTSYNISITTVSCGKMAAPTRNTCTTGITDPPPPDGSPNITSVSHNSVKVKFSGFEASHGPIKAYAVILTTGEAGHPSADVLKYTYEDFKKGASDTYVTYLIRTEEKGRSQSLSEVLKYEIDVGNESTTLGYYNGKLEPLGSYRACVAGFTNITFHPQNKGLIDGAESYVSFSRYSDAVSLPQDPGVICG

SEQ ID NO: 7 - DNA, 279 bp, Artificial Sequence
  CD45 Fibronectin type-III
agtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgct

SEQ ID NO: 8 - protein, 93 aa, Artificial Sequence
  CD45 Fibronectin type-III
SPGEPQIIFCRSEAAHQGVITWNPPQRSFHNFTLCYIKETEKDCLNLDKNLIKYDLQNLKPYTKYVLSLHAYIIAKVQRNGSAAMCHFTTKSA

SEQ ID NO: 9 - DNA, 279 bp, Artificial Sequence
  CD45 Fibronectin type-III 2
cctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattct

SEQ ID NO: 10 - protein, 93 aa, Artificial Sequence
  CD45 Fibronectin type-III 2
PPSQVWNMTVSMTSDNSMHVKCRPPRDRNGPHERYHLEVEAGNTLVRNESHKNCDFRVKDLQYSTDYTFKAYFHNGDYPGEPFILHHSTSYNS

SEQ ID NO: 11 - protein, 540 aa, Artificial Sequence
  CD2 Ig-like V-type domain + Ig-like C2-type domain
AAAGAGATTACGAATGCCTTGGAAACCTGGGGTGCCTTGGGTCAGGACATCAACTTGGACATTCCTAGTTTTCAAATGAGTGATGATATTGACGATATAAAATGGGAAAAAACTTCAGACAAGAAAAAGATTGCACAATTCAGAAAAGAGAAAGAGACTTTCAAGGAAAAAGATACATATAAGCTATTTAAAAATGGAACTCTGAAAATTAAGCATCTGAAGACCGACGATCAGGATATTTACAAGGTATCAATAGATGATACAAAAGGAAAAAATGTGTTGGAAAAAATATTTGATTTGAAGATTCAAGAGAGGGTCTCAAAACCAAAGATTTCCTGGACTTGTATCAACACAACCCTGACCTGTGAGGTAATGAATGGAACTGACCCCGAATTAAACCTGTATCAAGATGGGAAACATCTAAAACTTTCTCAGAGGGTCATCACACACAAGTGGACCACCAGCCTGAGTGCAAAATTCAAATGCACAGCAGGGAACAAAGTCAGCAAGGAATCCAGTGTCGAGCCTGTCAGCTGTCCC

SEQ ID NO: 12 - protein, 180 aa, Artificial Sequence
  CD2 Ig-like V-type domain + Ig-like C2-type domain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCP

SEQ ID NO: 13 - DNA, 291 bp, Artificial Sequence
  CD22 Ig-like C2-type 6 domain
ccgtcgacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcg

SEQ ID NO: 14 - protein, 94 aa, Artificial Sequence
  CD22 Ig-like C2-type 6 domain
LEVLYAPRRLRVSMSPGDQVMEGKSATLTCESDANPPVSHYTWFDWNNQSLPYHSQKLRLEPVKVQHSGAYWCQGTNSVGKGRSPLSTLTVYYS

SEQ ID NO: 15 - DNA, 537 bp, Artificial Sequence
  CD22 Ig-like C2-type 6 domain and CD22 Ig-like C2-type 5 domain
ccgtcgaccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcg

SEQ ID NO: 16 - protein, 179 aa, Artificial Sequence
  CD22 Ig-like C2-type 6 domain and CD22 Ig-like C2-type 5 domain
PSTPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYS

SEQ ID NO: 17 - DNA, 795 bp, Artificial Sequence
  CD22 Ig-like C2-type 6 domain and CD22 Ig-like C2-type 5 domain and CD22 Ig-like C2-type 4 domain
ccgtcgacccccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcg

SEQ ID NO: 18 - protein, 265 aa, Artificial Sequence
  CD22 Ig-like C2-type 6 domain and CD22 Ig-like C2-type 5 domain and CD22 Ig-like C2-type 4 domain
PSTPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYS

SEQ ID NO: 19 - DNA, 1056 bp, Artificial Sequence
  CD22 Ig-like C2-type 6 domain and CD22 Ig-like C2-type 5 domain and CD22 Ig-like C2-type 4 domain and CD22 Ig-like C2-type 3 domain
ccgtcgaccgaaccttccacggttcagatcctccactcaccggctgtggagggaagtcaagtcgagtttctttgcatgtcactggccaatcctcttccaacaaattacacgtggtatcacaatgggaaagaaatgcagggaaggacagaggagaaagtccacatcccaaagatcctcccctggcacgctgggacttattcctgtgtggcagaaaacattcttggtactggacagaggggaccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcg

SEQ ID NO: 20 - protein, 352 aa, Artificial Sequence
  CD22 Ig-like C2-type 6 domain and CD22 Ig-like C2-type 5 domain and CD22 Ig-like C2-type 4 domain and CD22 Ig-like C2-type 3 domain
PSTEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYS

SEQ ID NO: 21 - DNA, 1326 bp, Artificial Sequence
  CD22 Ig-like C2-type 6 domain and CD22 Ig-like C2-type 5 domain and CD22 Ig-like C2-type 4 domain and CD22 Ig-like C2-type 3 domain and CD22 Ig-like C2-type 2 domain
ccgtcgaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaaatactgctgtcaggtctccaatgacgtgggaccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtatcacaatgggaaagaaatgcagggaaggacagaggagaaagtccacatcccaaagatcctcccctggcacgctgggacttattcctgtgtggcagaaaacattcttggtactggacagaggggaccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcg

SEQ ID NO: 22 - protein, 442 aa, Artificial Sequence
  CD22 Ig-like C2-type 6 domain and CD22 Ig-like C2-type 5 domain and CD22 Ig-like C2-type 4 domain and CD22 Ig-like C2-type 3 domain and CD22 Ig-like C2-type 2 domain
PSTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYS

SEQ ID NO: 23 - DNA, 1632 bp, Artificial Sequence
  CD22 Ig-like C2-type 6 domain and CD22 Ig-like C2-type 5 domain and CD22 Ig-like C2-type 4 domain and CD22 Ig-like C2-type 3 domain and CD22 Ig-like C2-type 2 domain and CD22 Ig-like C2-type 1 domain
ccgtcgacaccttttccacctcatatccagctccctccagaaattcaagagtcccaggaagtcactctgacctgcttgctgaatttctcctgctatgggtatccgatccaactccagtggctcctagagggggttccaatgaggcaggctgctgtcacctcgacctccttgaccatcaagtctgtcttcacccggagcgagttgaagttctccccacagtggagtcaccacgggaagattgtgacctgccagcttcaggatgcagatgggaagttcctctccaatgacacggtgcagctgaacgtgaagcacaccccgaagttggagatcaaggtcactcccagtgatgccatagtgagggagggggactctgtgaccatgacctgcgaggtcagcagcagcaacccggagtacacgacggtatcctggctcaaggatgggacctcgctgaagaagcagaatacattcacgctaaacctgcgcgaagtgaccaaggaccagagtgggaaatactgctgtcaggtctccaatgacgtgggaccgggaaggtcggaagaagtgttcctgcaagtgcagtatgccccggaaccttccacggttcagatcctccactcaccggctgtggagggaagtcaagtcgagtttctttgcatgtcactggccaatcctcttccaacaaattacacgtggtatcacaatgggaaagaaatgcagggaaggacagaggagaaagtccacatcccaaagatcctcccctggcacgctgggacttattcctgtgtggcagaaaacattcttggtactggacagaggggaccgggagctgagctggatgtccagtatcctcccaagaaggtgaccacagtgattcaaaaccccatgccgattcgagaaggagacacagtgaccctttcctgtaactacaattccagtaaccccagtgttacccggtatgaatggaaacctcatggggcctgggaggagccatcgcttggggtgctgaagatccaaaacgtaggctgggacaacacaaccatcgcctgcgcagcttgtaatagttggtgctcttgggcctcccctgtcgccctgaatgtccagtatgccccccgagacgtgagggtccggaaaatcaagcccctttccgagattcactctggaaactcggtcagcctccaatgtgacttctcaagcagccaccccaaagaagtccagttcttctgggagaaaaatggcaggcttctggggaaagaaagccagctgaattttgactccatctccccagaagatgctgggagttacagctgctgggtgaacaactccataggacagacagcgtccaaggcctggacacttgaagtgctgtatgcacccaggaggctgcgtgtgtccatgagccctggggaccaagtgatggaggggaagagtgcaaccctgacctgtgagagcgacgccaaccctcccgtctcccactacacctggtttgactggaataaccaaagcctcccctaccacagccagaagctgagattggagccggtgaaggtccagcactcgggtgcctactggtgccaggggaccaacagtgtgggcaagggccgttcgcctctcagcaccctcaccgtctactactcg

SEQ ID NO: 24 - protein, 544 aa, Artificial Sequence
  CD22 Ig-like C2-type 6 domain and CD22 Ig-like C2-type 5 domain and CD22 Ig-like C2-type 4 domain and CD22 Ig-like C2-type 3 domain and CD22 Ig-like C2-type 2 domain and CD22 Ig-like C2-type 1 domain
PSTPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYS

SEQ ID NO: 25 - DNA, 78 bp, Artificial Sequence
  CD2 transmembrane domain
atctatctcatcattgggatatgtggaggaggcagcctcttgatggtctttgtggcactgctcgttttctatatcacc

SEQ ID NO: 26 - protein, 26 aa, Artificial Sequence
  CD2 transmembrane domain
IYLIIGICGGGSLLMVFVALLVFYIT

SEQ ID NO: 27 - DNA, 66 bp, Artificial Sequence
  CD4 transmembrane domain
atggccctgattgtgctggggggcgtcgccggcctcctgcttttcattgggctaggcatcttcttc

SEQ ID NO: 28 - protein, 22 aa, Artificial Sequence
  CD4 transmembrane domain
MALIVLGGVAGLLLFIGLGIFF

SEQ ID NO: 29 - DNA, 63 bp, Artificial Sequence
  CD8  transmembrane domain
atctacatctgggcgcccttggccgggacttgtggggtccttctcctgtcactggttatcacc

SEQ ID NO: 30 - protein, 21 aa, Artificial Sequence
  CD8  transmembrane domain
IYIWAPLAGTCGVLLLSLVIT

SEQ ID NO: 31 - DNA, 63 bp, Artificial Sequence
  CD45 transmembrane domain
gcactgatagcatttctggcatttctgattattgtgacatcaatagccctgcttgttgttctc

SEQ ID NO: 32 - protein, 21 aa, Artificial Sequence
  CD45 transmembrane domain
ALIAFLAFLIIVTSIALLVVL

SEQ ID NO: 33 - DNA, 63 bp, Artificial Sequence
  CD148 transmembrane domain
gcggtttttggctgtatctttggtgccctggttattgtgactgtgggaggcttcatcttctgg

SEQ ID NO: 34 - protein, 21 aa, Artificial Sequence
  CD148 transmembrane domain
AVFGCIFGALVIVTVGGFIFW

SEQ ID NO: 35 - DNA, 351 bp, Artificial Sequence
  CD2 intracellular domain
aaaaggaaaaaacagaggagtcggagaaatgatgaggagctcgaaacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagaccacgggttcagccaaaacctccccacggggcagcagaaaactcattgtccccttcctctaattaa

SEQ ID NO: 36 - protein, 116 aa, Artificial Sequence
  CD2 intracellular domain
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 37 - DNA, 2127 bp, Artificial Sequence
  CD45 intracellular domain
tacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcatag

SEQ ID NO: 38 - protein, 708 aa, Artificial Sequence
  CD45 intracellular domain
YKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 39 - DNA, 1026 bp, Artificial Sequence
  CD148 intracellular domain
agaaagaagaggaaagatgcaaagaataatgaagtgtccttttctcaaattaaacctaaaaaatctaagttaatcagagtggagaattttgaggcctacttcaagaagcagcaagctgactccaactgtgggttcgcagaggaatacgaagatctgaagcttgttggaattagtcaacctaaatatgcagcagaactggctgagaatagaggaaagaatcgctataataatgttctgccctatgatatttcccgtgtcaaactttcggtccagacccattcaacggatgactacatcaatgccaactacatgcctggctaccactccaagaaagattttattgccacacaaggacctttaccgaacactttgaaagatttttggcgtatggtttgggagaaaaatgtatatgccatcattatgttgactaaatgtgttgaacagggaagaaccaaatgtgaggagtattggccctccaagcaggctcaggactatggagacataactgtggcaatgacatcagaaattgttcttccggaatggaccatcagagatttcacagtgaaaaatatccagacaagtgagagtcaccctctgagacagttccatttcacctcctggccagaccacggtgttcccgacaccactgacctgctcatcaacttccggtacctcgttcgtgactacatgaagcagagtcctcccgaatcgccgattctggtgcattgcagtgctggggtcggaaggacgggcactttcattgccattgatcgtctcatctaccagatagagaatgagaacaccgtggatgtgtatgggattgtgtatgaccttcgaatgcataggcctttaatggtgcagacagaggaccagtatgttttcctcaatcagtgtgttttggatattgtcagatcccagaaagactcaaaagtagatcttatctaccagaacacaactgcaatgacaatctatgaaaaccttgcgcccgtgaccacatttggaaagaccaatggttacatcgcctaa

SEQ ID NO: 40 - protein, 341 aa, Artificial Sequence
  CD148 intracellular domain
RKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 41 - DNA, 1038 bp, Artificial Sequence
  Notch core regulatory region of SynNotch receptor
tcgagcatcctggactacagcttcgggggtggggccgggcgcgacatccccccgccgctgatcgaggaggcgtgcgagctgcccgagtgccaggaggacgcgggcaacaaggtatgcagcctccagtgcaacaaccacgcctgcggctgggacggcggtgactgctccctcaacttcaatgacccctggaagaactgcacgcagtctctccagtgctggaaatacttcagtgacggccactgtgacagccagtgcaactcagccggctgcctcttcgacggctttgactgccagcgtgcggaaggccagtgcaaccccctgtacgaccaatactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctaagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccctatggacgtgcgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcaattgcacttcatgtacgtggcggcagccgcctttgtgcttctgttcttcgtgggctgcgggaaactgctgtcccgccggcgacggcggcagcatggccagctctggttccct

SEQ ID NO: 42 - protein, 346 aa, Artificial Sequence
  Notch core regulatory region of SynNotch receptor
SSILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGKLLSRRRRRQHGQLWFP

SEQ ID NO: 43 - DNA, 804 bp, Artificial Sequence
  anti-HLA-A2 scFV
atgaagttgcctgttaggctgttggtgctgatgttctggattcctgcttccagcagtgatgttttgatgacccaaactccactctccctgcctgtcagtcttggagatcaagtctccatctcttgcagatccagtcagagcatcgtacatagtaatggaaacacctatttagaatggtatctccagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcccatgttcctcggacgttcggtggaggcaccaagctggaaatcaaacggggatcaacttcgggcagtggtaagcctggtagtggtgagggtagtaccaagggccaggtccagctccagcagtctggacctgagctggtgaagcctggggcctcagtgaagatgtcctgcaaggcttctggctacaccttcacaagctaccatatacagtgggtgaagcagaggcctggacagggacttgagtggattggatggatttatcctggagatggtagtacccagtacaatgagaagttcaagggcaagaccacactgaccgcagacaaatcctccagcacagcctacatgctgctcagcagcctgacctctgaggactctgcgatctatttctgtgcaagggaggggacctactatgctatggactactggggtcaaggaacctcagtcaccgtctcctca

SEQ ID NO: 44 - protein, 268 aa, Artificial Sequence
  anti-HLA-A2 scFV
MKLPVRLLVLMFWIPASSSDVLMTQTPLSLPVSLGDQVSISCRSSQSIVHSNGNTYLEWYLQKPGQSPKLLIYKVSNRFSGVPDRFSGSGSGTDFTLKISRVEAEDLGVYYCFQGSHVPRTFGGGTKLEIKRGSTSGSGKPGSGEGSTKGQVQLQQSGPELVKPGASVKMSCKASGYTFTSYHIQWVKQRPGQGLEWIGWIYPGDGSTQYNEKFKGKTTLTADKSSSTAYMLLSSLTSEDSAIYFCAREGTYYAMDYWGQGTSVTVSS

SEQ ID NO: 45 - protein, 100 aa, Artificial Sequence
  iCAR with CD2 mutant endodomains
TRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSS

SEQ ID NO: 46 - protein, 100 aa, Artificial Sequence
  iCAR with CD2 mutant endodomains
TRAHRVATEERGRKPHQIPASTPQNPATSQHPPAAAGHRSQAPSHRPAAAGHRVQHQPQKRPPAPSGTQVHQQKGPPLPRPRVQPKPPHGAAENSLSPSS

SEQ ID NO: 47 - protein, 100 aa, Artificial Sequence
  iCAR with CD2 mutant endodomains
TRAHRVATEERGRKPHQIPASTPQNPATSQHPPPPPGHRSQAPSHRPPPPGHRVQHQPQKRPPAPSGTQVHQQKGAALARARVQAKAAHGAAENSLSPSS

SEQ ID NO: 48 - protein, 100 aa, Artificial Sequence
  iCAR with CD2 mutant endodomains
TRAHRVATEERGRKPHQIPASTPQNPATSQHPPAAAGHRSQAPSHRPAAAGHRVQHQPQKRPPAPSGTQVHQQKGAALARARVQAKAAHGAAENSLSPSS

SEQ ID NO: 49 - protein, 32 aa, Artificial Sequence
  Mouse Notch1 (UniProtKB Q01705)
PSQLHLMYVAAAAFVLLFFVGCGVLLSRKRRR

SEQ ID NO: 50 - protein, 32 aa, Artificial Sequence
  Human Notch1 (UniProtKB P46531)
PAQLHFMYVAAAAFVLLFFVGCGVLLSRKRRR

SEQ ID NO: 51 - protein, 32 aa, Artificial Sequence
  Mutated Human Notch 1 (35)
PAQLHFMYVAAAAFVYLFFVGCGVLLSEKRRR